(19) United States
(12) Patent Application Publication
(10) Pub. No.: US 2008/0312162 A1
(43) Pub. Date: Dec. 18, 2008

(54) Methods and Compositions for Enzyme-Specific Activation of Carbohydrate-Conjugated Prodrugs

(75) Inventors: Duxin Sun, Dublin, OH (US); Peng George Wang, Columbus, OH (US); Edward Martin, JR., Delaware, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY, Columbus, OH (US)

(21) Appl. No.: 11/664,034

(22) PCT Filed: Oct. 7, 2005
(86) PCT No.: PCT/US05/36061

§ 371 Date: Jul. 8, 2008

Related U.S. Application Data
(60) Provisional application No. 60/617,390, filed on Oct. 8, 2004.

(51) Int. Cl. A61K 31/70; A61P 35/04

(52) U.S. Cl. 514/23

(57) ABSTRACT

Methods for the targeted activation of prodrugs by enzymes, which cleave a linkage between a carbohydrate conjugate and a drug. Means to target the activation of prodrugs to specific cells by linking the enzyme to an antibody molecule. Carbohydrate conjugates of geldanamycin.

This application claims priority to U.S. Provisional Application No. 60/617,390, filed Oct. 8, 2004, the entire disclosure of which is incorporated herein by reference.

Generally, the invention relates to methods of synthesizing and activating prodrugs. Methods of use of these prodrugs are also provided. In some embodiments, prodrugs comprise carbohydrate conjugates.

Adjuvant chemotherapy is the most common treatment for cancer. Tremendous effort has been expended to improve the efficacy of chemotherapy. Yet chemotherapy is only partially successful at treating cancer. Chemotherapy is often dose- and toxicity-limited. A delicately balanced dose regimen is usually required to regulate drug toxicity and resistance. A high dose might cause significant tissue toxicity while only partially controlling neoplastic growth. A low dose might limit tissue toxicity but induce drug resistance in the neoplasm because of marginal effects on neoplastic growth. Therefore, a targeted drug delivery system to increase drug efficacy and decrease drug toxicity will be beneficial for cancer chemotherapy.

The development of new anti-neoplastic agents has long been hindered by a number of factors, including, for example, limited aqueous solubility of drug agents and the general toxicity of agents with cytostatic or anti-neoplastic activity. One example of such a drug agent is geldanamycin. Geldanamycin (GA) is a compound with anticytostatic properties and is a potent anticancer antibiotic, but its potential clinical utility is hampered by severe hepatotoxicity. Geldanamycin is produced by fermentation of the microbe Streptomyces hygroscopicus. It is a benzoquinoid ansamycin related to herbimycin A and macbecin (FIG. 1, Scheme 1). Geldanamycin was initially thought to be a nonspecific kinase inhibitor, but is now believed to target the heat shock protein 90 (Hsp90).

Hsp90 is a molecular chaperon known to modulate protein kinase activity (such as p60, p185, raf-1, cdk4/cdk6, FAK, MAK, v-Src, Akt, Bcr-Abl, mutant P53, HIF-1α, and ErbB2) in cells. Overexpression of Hsp90 has been observed in various human cancers, and Hsp90 is expressed at 2 to 10-fold higher levels in tumor cells compared to normal cells. Crystal structure determinations show GA binds Hsp90 and inhibits Hsp90-mediated protein conformational/refolding, resulting in a depletion of oncogenic kinases through the proteasomic degradation of immature proteins. This process subsequently down-regulates expression of many oncogenes in cancer cells.

For further background on geldanamycin and Hsp90, see:
(1) DeBoer, C.; Meulman, P. A.; Wnuk, R. J.; Peterson, D. H. “Geldanamycin, a new antibiotic.” Journal of Antibiotics 23: 442-447 (1970).
(2) Ferrarini, M., Heltai, S., Zocchi, M. R., Rugarli, C., “Unusual expression and localization of heat-shock proteins in human tumor cells.” International Journal of Cancer., 51: 613-619 (1992).
(3) Stebbins, C. E., Russo, A. A.; Schneider, C., Rosen, N., Hartl, F. U. et al. “Crystal structure of an Hsp90-geldanamycin complex: targeting of a protein chaperone by an antitumor agent.” Cell 89: 239-250 (1997).
(4) Neckers, L., Schulte, T. W., Mimnaugh, E. “Geldanamycin as a potential anti-cancer agent: its molecular target and biochemical activity.” Investigational new drugs. 17: 361-373 (1999).

Most existing chemotherapeutic compounds used in antineoplastic therapy are limited by low tumor selectivity. When chemotherapeutic compounds lack selectivity, the same compounds with antineoplastic activity may also have a cytostatic effect on other cells and, thus, have associated dose-limiting side effects. While the antineoplastic potential of geldanamycin has long been recognized, clinical use of native GA has not been pursued because of low selectivity, in that tissues other than the target tissue are damaged by the drug. For instance, one effect of the low selectivity of geldanamycin is severe hepatotoxicity (liver damage). In addition, GA has poor aqueous solubility, making drug delivery to target cells difficult.

A number of chemical analogues have been created in efforts to modify GA and increase clinical efficacy. One such analog, 17-allylaminogeldanamycin (17-AAG), has entered phase II clinical trials at the National Cancer Institute. Although 17-AAG showed improved efficacy and lower generalized toxicity, hepatotoxicity and low aqueous solubility were still limiting factors for clinical application.

For further background on geldanamycin analogs see:
(5) Supko, J. G., Hickman, R. L., Grever, M. R., Malspeis, L., “Preclinical pharmacologic evaluation of geldanamycin as an antitumor agent.” Cancer Chemother Pharmacol. 36: 305-315 (1995).
(6) P. Munster et al., Modulation of Hsp90 function by ansamycins sensitizes breast cancer cells to chemotherapy-induced apoptosis in an RB- and schedule-dependent manner. Clin. Cancer Res. 7: 2228-2236 (2001).
(7) Sausville, E. A. Commentary “Combining cytotoxics and 17-allylamino, 17-demethoxygeldanamycin: sequence and tumor biology matters.” Clin.

Cancer Res. 7: 2155-2158 (2001).
(8) Schnur, R. C., Corman, M. L., Gallaschun, R. J., Cooper, B. A., Dee, M. F. et al. “erbB-2 oncogene inhibition by geldanamycin derivatives: synthesis, mechanism of action, and structure-activity relationships.” Journal of Medicinal Chemistry 38: 3813-3820 (1995).
(9) Adams, J., Elliott, P. J. “New agents in cancer clinical trials.” Oncogene 19: 6687-6692 (2000).
(10) Egorin, M. J., Zuhowski, E. G., Rosen, D. M., Sentz, D. L., Covey, J. M. et al. “Plasma pharmacokinetics and tissue distribution of 17-(allylamino)-17-demethoxygeldanamycin (NSC 330507) in CD2F1 mice1.” Cancer Chemother. Pharmacol. 47: 291-302 (2001).
(11) Kelland, L. R., Sharp, S. Y., Rogers, P. M., Myers, T. G., Workman, P. “DT-Diaphorase expression and tumor cell sensitivity to 17-allylamino, 17-demethoxygeldanamycin, an inhibitor of heat shock protein 90.” J. Natl. Cancer Inst. 91: 1940-1949 (1999).

In other cases, cytotoxic compounds are linked to monoclonal antibodies against tumor-specific antigen in an attempt to increase specific activity. However, this approach is limited by the amount of drugs that can be linked to antibodies, the slow internalization of drug conjugates into targeted cancer cells, and heterogeneous antigen expression even within the cells of single solid tumors. To circumvent these problems, a two-step strategy called antibody-directed enzyme prodrug therapy (ADEPT) has recently emerged. In this approach, a monoclonal antibody is employed to selectively deliver enzyme, such as beta-galactosidase or alpha-galactosidase, to the tumor cells, which subsequently converts an inactive prodrug to active drug. Thus, the selective activation of the prodrug at the tumor site results in improved antitumor activity with minimal side effects.

In ADEPT therapy, the first step is administration of antibody-enzyme complexes that accumulate in tumor cells, followed by the second step where an inactive prodrug is provided to the patient. The low-toxicity prodrug is then converted into a cytotoxic drug by enzymatic activation in the targeted tumor tissues. Many combinations of tumor targeting antibodies, prodrug activating enzymes, and prodrugs have been tested in this approach as summarized in Table 1.

TABLE 1
Summary of enzymes, antibodies and drugs utilized in ADEPT

| Enzyme | Antibody | Drug |
|---|---|---|
| Alkaline phosphatase | HMFG1 | doxorubicin |
| Aminopeptidase | H17E2 | Mitomycin C |
| Aryl sulfatase | ING-1 | A vinca alkaloid |
| Carboxypeptidase A | Recomb. MHC II | paclitaxel |
| Carboxypeptidase G2 | mAb BR96 | 5-fluorouracil |
| Cytosine deaminase | Anti-CEA | cisplatin |
| α-Galactosidase | Anti-TAG-72 | daunorubicin |
| β-Galactosidase | L6 | methotrexate |
| β-Glucosidase | 1F5 | SAHA |
| β-Glucuronidase | anti-sialamucin | |
| β-Lactamase | | |
| Histone deacetylase | | |
| Nitroreductase | | |
| Penicillin amidase | | |

An ongoing difficulty with ADEPT is identification of an antibody effective for targeted activation and identification of appropriate enzymes for prodrug activation. Ideally, a tumor-specific antigen would exhibit homogeneous expression in a broad range of tumors thus allowing the antibody to target many different cancers or most of a certain type of cancer. The enzymes utilized in ADEPT need to be screened by methods well-known to those skilled in immunology for minimal immunogenicity in the patient. The enzyme needs to be absent or present in reduced concentrations in the plasma or body fluids that are not targeted by the activated prodrug.

One antibody that is applicable to ADEPT is anti-TAG-72. Tumor-associated antigen (TAG-72) is a human mucin (MUC1)-like glycoprotein complex with molecular weight of 10⁶ Da. It is overexpressed in several epithelial-derived cancers, including most ductal carcinomas of the breast, common epithelial ovarian carcinomas, non-small cell lung carcinomas, gastric, pancreatic, and colorectal carcinomas. Murine monoclonal antibody (B72.3) was generated using membrane-enriched extracts of human metastatic mammary carcinoma lesions, while the second generation monoclonal antibody (CC49) was generated against purified TAG-72 from colon cancer. These antibodies have been extensively evaluated in animal models and human for detection of various cancers, one of which has been approved by FDA for the detection of both colorectal and ovarian cancers with in gamma camera scanning in conjunction with computerized tomography (¹¹¹Indium-labeled B72.3 antibody, CYT-103, Cytogen).

TAG-72 antibody shows selective reactivity for human carcinomas, demonstrating that 94% of colon carcinomas cancer express the TAG-72, while normal colon epithelium does not show any reactivity to the antibody. Murine monoclonal B72.3 also reacted with cells in areas of “atypia” within adenomas. It also showed reactivity with other human carcinomas including 84% of invasive ductal breast cancer, 100% of ovarian cancers tested, and 96% lung of adenocarcinomas, while it showed only weak or no reactivity in the corresponding normal tissues except secretory endometrium.

B72.3 antibody has been evaluated in tissue culture and xenograft models. Interestingly, this antibody is not reactive to the vast majority of human carcinoma cell lines in cultures due to limitations in this special configuration. However, it is highly expressed in colon cancer cell lines (e.g., LS 174T) and breast cancer cells lines (e.g., MCF-7). When these cells were grown in spheroid culture, suspension cultures, or on agar, TAG-72 expression increased by 2-10 fold. In addition, when the LS 174T cell line was injected into athymic mice to generate xenograft models, the level of TAG-72 antigen increased over 100-fold, which is similar to expression levels seen in the metastatic tumor masses from patients. I¹²⁵-labeled B72.3 was tested in xenograft mice models with LS-174 cancer cells for tumor localization. After intravenous injection of 1.5 μCi of ¹²⁵I-labeled B72.3, 10% of injected dose per gram of body weight (% ID/g) was determined after two days. Interestingly, the total amount of ¹²⁵I-B72.3 activity in the tumor stayed constant during 30 days, while the activity in the rest of the body including blood, kidney, liver, spleen, and lung decreased significantly. For example, the % ID/g of 125I-B72.3 in tumors stayed at 6.49% to 10.75% in 7 days period, while it decreased from 9.94% to 1.38% in blood, 1.82% to 0.34% in kidney, 2.23% to 0.37% in spleen, 5.52% to 0.75% in lung, and 1.89% to 0.37%. The distribution ratio of tumor compared to other normal organs (liver, spleen, kidney, lung) reached 18:1 at day 7, while tumor to blood ratio reached 5:1 at day 7. In xenograft models with A375 cells without TAG-72 expression, B72.3 did not show any tumor localization. In xenograft models implanting LS 174T with high levels of TAG-72, other control antibodies such as ¹²⁵I-MOPC-21 IgG did not show tumor localization either.

¹³¹I-labeled B72.3 IgG has been used clinically for diagnostic imaging of colorectal, ovary, and breast cancer. The data demonstrates the specific localization of B72.3 antibody in cancer tissues in patients. After intravenously administered ¹³¹I-labeled B72.3 IgG prior to surgery, radio-localization indices (RI) were calculated by cpm of ¹³¹I-labeled antibody per gram of tumor versus cpm per gram of normal tissues. Seventy percent (99 of 142) of tumor lesions showed RI is of greater than 3 (antibody localization in tumors is 3 times greater than normal tissue). In addition, high-performance liquid chromatography (HPLC) and SDS-polyacrylamide gel electrophoresis demonstrated that the radioactivity in patient's sera was associated with intact ¹³¹I-B72.3 antibody as visualized in autoradiography or IgG peak in HPLC analysis after IV administration of dose range 0.5-20 mg (48). Interestingly, when ¹³¹I-labeled B72.3 IgG was administered intraperitoneally in colon cancer patients, the localization in colon tumor verse normal tissue was 70:1. However, i.v. administration of this labeled antibody is more efficient in targeting lymph node metastases.

¹³¹I- or ¹²⁵I-labeled B72.3 has also been used for radio-immunoguided surgery (RIGS) with an intraoperative hand-held probe to localize the residual tumor tissue for resection. RIGS has also been successfully used with the B72.3 antibody for clinical colorectal cancer patients. ¹²⁵I-labeled-antibody has localized 75-80% of primary colorectal tumor lesion, and 63-73% of metastatic lesions in lymph nodes and liver.

The second generation antibody CC49 was generated against TAG-72 purified from colon cancer. CC49 showed higher binding affinity than B72.3 to TAG-72 in carcinomas including breast, colorectal, ovarian, and lung carcinomas, while CC49 exhibited minimum reactivity with normal tissues. When ¹²⁵I-CC49 was administered in xenograft models with colon cancer cells LS 174T, the plasma clearance was much faster than B72.3, which results in much higher tumor to normal tissue distribution ratio. For example, the tumor to blood ratio was 18.1, tumor to liver ratio 3.81, tumor to spleen ratio 16.64, tumor to kidney ratio 36.48, and tumor to lung ratio 25.82. In RIGS studies of 300 patients with colorectal cancers, CC49 was able to successfully detect tumors in 86% of patients with primary tumors and 95% of patients with recurrent tumors. In addition, clinical studies of a modified humanized antibody CC49ΔC_{H}2 with a deletion in glycosylation sites of the antibody showed similar results with CC49 in detection of colorectal cancer.

Both B72.3 and CC49 have demonstrated promising results in tumor detection utilizing the RIGS procedure to significantly improve patient survival rate. However, in many cases, patients have shown metastatic cancers or multiple lesions which are not resectable. In such cases, even though the antibodies used with RIGS are able to detect the tumors, surgery cannot be employed to remove the tumors. Embodiments of the invention overcome this limitation by combining RIGS based tumor detection with site-specific activation of prodrugs using a TAG-72 antibody-enzyme complex. The invention provides a new cancer therapy especially for the non-resectable, metastatic, or residual tumors.

For further background on antigen expression and antibody-directed enzyme prodrug therapy see:
(12) Jain, R. K. “Physiological barriers to delivery of monoclonal antibodies and other macromolecules in tumors.” Cancer Res, 50: 814s-819s (1990).
(13) Sung, C., Dedrick, R. L., Hall, W. A., Johnson, P. A., Youle, R. J. “The spatial distribution of immunotoxins in solid tumors: assessment by quantitative autoradiography.” Cancer Res, 53, 2092-2099 (1993).
(14) Senter, P. D., Saulnier, M. G., Schreiber, G. J., Hirschberg, D. L., Brown, J. P., et al. “Anti-tumor effects of antibody-alkaline phosphatase conjugates in combination with etoposide phosphate.” Proc. Natl. Acad. Sci. USA 85: 4842-4846 (1988).
(15) Bagshawe, K. D. “Antibody directed enzymes revive anti-cancer prodrugs concept.” British Journal of Cancer 56: 531-532 (1987).
(16) Sherwood, R. F. “Advanced drug delivery reviews: enzyme prodrug therapy.” Advanced Drug Delivery Reviews 22: 269-288 (1996).

Thus, the development of methods to target drug delivery with greater specificity to neoplastic cells will enhance the effectiveness of anti-neoplastic treatments.

The present invention relates to development of new antineoplastic drugs and the targeting of drugs to specific targets in the treatment of cancer and other diseases and more particularly to a technique for creating prodrugs by conjugating a carbohydrate to an active drug and a technique for targeted enzymatic activation of prodrugs.

### BRIEF DESCRIPTION OF THE DRAWINGS

FIG. 1 (Scheme 1) shows a structural diagram of geldanamycin.

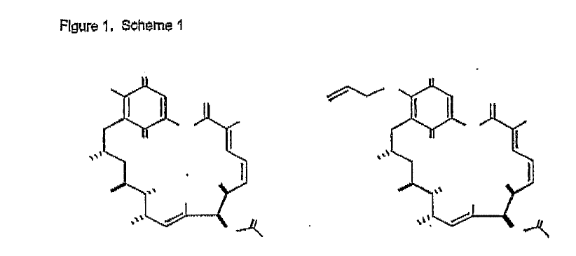

FIG. 2 is a graph showing anticancer activity of carbohydrate-geldanamycin conjugates (24-29) tested in the SW620 cell line as a function of drug concentration in nanomoles/liter versus relative percentage of cell growth.

FIG. 3 is a graph showing anticancer activity of carbohydrate-geldanamycin conjugates (24-29) tested in the HT29 cell line as a function of drug concentration in nanomoles/liter versus relative percentage of cell growth.

FIG. 4 is a graph showing anticancer activity of carbohydrate-geldanamycin conjugates (24-29) tested in the K562 cell line as a function of drug concentration in nanomoles/liter versus relative percentage of cell growth.

FIG. 5 is a graph showing beta-glucosidase inhibition assay of carbohydrate-geldanamycin conjugates 24- and 25 studied in K562 cell line as a function of treatment composition versus relative percentage of cell growth.

FIG. 6 is a graph showing beta-galactosidase cleavage assay of carbohydrate-geldanamycin conjugates (24-29) and GA in the SW620 cell line as a function of treatment composition versus relative percentage of cell growth.

FIG. 7 is a graph-showing beta-galactosidase cleavage assay of carbohydrate-geldanamycin conjugates (24-29) and GA in the HT 29 cell line as a function of treatment composition versus relative percentage of cell growth.

FIG. 8 is a graph showing beta-galactosidase cleavage assay of carbohydrate-geldanamycin conjugates (24-29) and GA in the K562 cell line as a function of treatment composition versus relative percentage of cell growth.

FIG. 9 (scheme 8) shows the synthesis procedure for synthesizing a self-elimination spacer.

FIG. 10 (Scheme 2) shows three trichloroacetimidate derivatives of sugar (galactose, glucose, and lactose) prepared as glycosyl donors from corresponding per-acetylated sugars.

FIG. 11 (Scheme 3) is a treatment of α-trichloroacetimidates (3, 5, 7) with different alcohol-linkers (8, 9, 10, 11) in the presence of TMSOTf.

FIG. 12 (Scheme 4) depicts six carbohydrate conjugates (24-29) with different sugar moieties or linkers.

FIG. 13 (Scheme 5) depicts syntheses resulting in inefficient glycosylation of geldanamycin at C11 position.

FIG. 14 (Scheme 6) shows direct linkage of glycosylamine to GA with the major product obtained being 17-amine-17-demethoxy-GA.

FIG. 15 is a graph showing antibody localization in patients from 3-21 days after antibody administration.

FIG. 16 is a graph showing composite precordial count vs. time curve for all patients in study.

FIG. 17 (scheme 7) shows the enzyme-antibody conjugation procedure.

The drawings will be described in further detail below.

The invention is directed to a method for creating carbohydrate-inactivated prodrugs, and for activating these prodrugs at a target location. The invention is applicable to treatment of a number of diseases of the human and animal body, but is particularly applicable to therapies designed to provide relief from neoplasms and cancers.

For purposes of this application the following definitions apply:

A patient is a subject to be treated for conditions associated with disease using any embodiment of the invention. As such, patients include subjects such as humans and other mammals and animals.

A pharmacophore is the group of atoms in a drug molecule that is responsible for the activity of the drug compound.

A prodrug is a compound that, upon administration, must undergo chemical conversion before becoming an active pharmacological agent (drug). The chemical conversion of a prodrug to an active drug can entail native metabolic processes, or be carried out by processes present in the patient, or the conversion could result from the action of a pharmacologic agent, or by any other chemical conversion.

An anti-neoplastic agent is a compound, protein, or other substance, which interferes with the growth, development, or homeostasis of neoplastic cells.

Neoplastic cells are cells that exhibit growth, development, or homeostasis resulting from genetic variation, such that the neoplastic cell grows in a manner that is identifiably different from the cells of a tissue from which the neoplasm originates.

Substrates for Synthesis of Prodrugs

A prodrug should have toxicity that is substantially less than that of the cytostatic agent, say, for example, about ten-fold less toxicity. For essentially all drug structure families, most structures will not have therapeutically effective activity. Therefore, inactivation of a drug can be accomplished by, for example, conjugation of a six-carbon saccharide to most drugs. Such a conjugated molecule would be efficacious as a prodrug if an enzyme activity can be identified that cleaves the conjugated saccharide and restores drug activity. Choice of a substrate for synthesizing a carbohydrate-inactivated prodrug would focus on identifying drug structures that have anti-cytostatic biological activity, and especially those drugs which are strongly toxic to cancer cells. A prodrug substrate should also have available functional groups that allow conjugation of inactivating carbohydrate groups. Because the specific target for many drugs is known, along with detailed structures of drugs and their target molecules, such information is highly useful for choosing functional groups to be subjected carbohydrate conjugation with the expectation of reversibly inactivating a drug.

Examples of active drugs that are predicted or known to serve as substrates for creating prodrugs by carbohydrate inactivation include, for example, one or more of paclitaxel, 9-aminocampthotecin, 5-fluorouracil, aniline-mustard, epirubicine, daunorubicin, doxorubicin, beta-naphthol, geldanamycin, cisplatin, nitrogen mustard, indolocarbazole, anthracyclines, SAHA, or rapamycin. These drugs characteristically have relatively high tissue toxicity, and thus drug dose needs to be carefully controlled in order to achieve maximal anti-cytostatic effect without causing disabling toxicity in the patient. The active drug substrates include, but are not limited, to anti-cytostatic agents, anticancer agents, antiviral agents, and antibiotics. Artisans will recognize that the choice of the inactivating conjugant and drug combination can modulate the chemical activity, solubility, availability, and/or stability of the prodrug.

The drug paclitaxel (Taxol) has exposed amino and hydroxyl groups that serve as available location for conjugating inactivating groups. Similarly, carbohydrates can be linked to the drug cisplatin, in order to inactivate it. A variation of the drug 5-fluorouracil (5-FU), known as Xeloda, is known in the art to possess reduced toxicity due to the addition of a modifying carbohydrate group. According to the invention, a larger, more strongly inactivating carbohydrate group can be attached with a linkage that can be cleaved by an identified enzyme, for instance a polysaccharide can be linked to the same functional group on 5-FU through an α-galactoside linkage, subject to cleavage by α-galactosidase.

The antineoplastic drug suberoylanilide hydroxamic acid (SAHA) is an anticancer compound that inhibits histone deacetylase (HDAc), and is believed to interfere with the active site of HDAc. Thus, conjugation of bulky carbohydrate groups to SAHA is expected to inactivate SAHA by interfering with its interactions with HDAc.

Chemical Synthesis of Carbohydrate Conjugated Prodrugs.

Prodrugs are generated from active drug substrates by conjugation of a carbohydrate moiety to one or more active pharmacophores of an active drug via either direct glycosylation or by means of intermediate labile linkers. The carbohydrates include, but are not limited to, mono-di-, tri-, or oligosaccharides.

Large inactivating groups of specific structure have advantages over single atom or functional group additions, because large specific structures will be expected to serve as the substrate for fewer enzymes, and, thus, release of the active drug will be limited by nonspecific activation of the prodrug. Specific carbohydrate groups that can be used to practice the invention include one or more of glucose, galactose, or lactose, and also one or more of mannose, fucose, n-acetylglucosamine, xylose, sialic acid, or glucuronic acid. In certain conditions, based on the stereochemistry of the drug target and the drug compound, it may be necessary to add multiple carbohydrates to a single functional group or to add carbohydrates to multiple functional groups.

Carbohydrates can be conjugated, for example, through direct linkage to pharmacophores, or through an intermediate linker structure. Linkers can be synthesized so that they are labile inside the patient, once the carbohydrate structure is removed by the activating enzyme. In addition, the linker can be synthesized to function as the substrate for an activating enzyme. In one embodiment of the invention, an acetate group can serve as a linker to a larger inactivating carbohydrate group, and the linker could cleaved by the activity of deacetylases known to those skilled in the art.

In addition, the sugar moiety is also expected to provide better water solubility for drug compounds, such as the drug geldanamycin. Many chemotherapeutic agents are relatively insoluble in the aqueous environment of the bloodstream and tissue fluids. Thus, the addition of a polar sugar moiety to the structure of a drug molecule provides increased solubility of the drug agent, and potentially improves delivery of the drug agent to target tissues. By increasing the aqueous solubility of a drug, not only can drug agents reach target tissues more rapidly through the bloodstream, but also a potentially a lower dose may be required to achieve the same concentration of drug in the target tissue. If the sugar moiety is cleaved from the carbohydrate conjugated prodrug at the target tissue location, then the advantages of improved solubility and transport, and lower total dose can be combined with full drug activity at the target tissue.

The choice of the carbohydrate structure conjugated to a drug molecule can provide a means to control the location of accumulation of a prodrug. For instance, conjugation of a small drug molecule to a carbohydrate for which there is a membrane transporter may allow, depending on the nature of the drug, increased accumulation of the prodrug inside cells. If a glucose molecule is linked to a small drug molecule, the glucose transporter of human cell membranes may continue to be capable of transporting the conjugated molecule into the cytoplasm. Those cells that have large glucose demands, such as cancer cells, would tend to accumulate the prodrug. Alternatively, conjugation of a large carbohydrate group for which there is no membrane transporter, could effectively exclude the conjugated prodrug from intracellular locations.

The inactivation of a wide variety of drugs by the conjugation of carbohydrate groups according to the invention provides a wide variety of chemotherapeutic agents that can be employed in targeted activation of chemotherapeutic agents at specific locations in a patient. The invention is embodied in novel carbohydrate-GA conjugates. No previous art shows GA-prodrugs in which GA is inactivated by conjugation of carbohydrate groups. In this regard, the invention embodies a synthetic program to produce prodrugs by conjugation of carbohydrate groups to new or existing drug structures, including carbohydrate-geldanamycin conjugates. With respect to GA, a bulky sugar structure can affect the binding of GA to Hsp90 and create an inactive prodrug. Other drugs that possess functional groups that serve as ready targets for carbohydrate conjugation according to the invention include 5-FU, cisplatin, paclitaxel, and SAHA. If the enzyme or chemical environment necessary to cleave the carbohydrate moiety from the conjugated prodrug is present inside or in the local environment of target cells, the drug will be activated at the target locations.

Enzymes

Design of a prodrug/enzyme pair can focus on creating an inactive prodrug, and then identifying an enzyme that will cleave the conjugating groups to release and active drug. The prodrug should be convertible into an active drug by an enzyme and the released drug agent should have a steep dose-activity curve, in that the released drug is highly active and, thus, even low activity of the enzyme can create sufficient active drug to kill target cells. Release of even low concentrations of active drug should ideally lead to rapid death of target cells, for example, tumor cells. Thus, development of resistance of tumor cells to the active drug will be limited or avoided.

For example, the enzyme alkaline phosphatase can be used to activate a phosphorylated prodrug by the removal of a single phosphate, but there are many endogenous alkaline phosphatases with low specificity that could activate the prodrug. Lower substrate specificity of an enzyme and or constitutive expression of an enzyme are expected to result in a reduction of drug target specificity and an increase in drug toxicity. More ideally, the activating enzyme should be absent in non-target tissues, be enriched in target tissues, such as beta-glucosidase, or be an enzyme variant that is absent for the tissues of the human patient such as beta-galactosidase.

An enzyme designed to activate a prodrug should be easily accessible by the substrate when present at the target site, have high substrate selectivity and turnover at 37° C. near neutral pH. The activation reaction should preferably be non-reversible.

Carbohydrate-inactivated prodrugs can be activated by enzymes such as, for example, one or more of acetylase, glucosidase, galactosidase, manosidase, glucoamylase, glucosaminidase, galactosaminidase, sialidase, xylosidase, or glucuronidase. Specific enzymes of the above general classes include, but are not limited to, alpha-glucosidase, beta-glucosidase, alpha-galactosidase, and beta-galactosidase.

Targeted Activation of Conjugated Prodrugs

For effective implementation of prodrug therapy, it is beneficial that a prodrug be activated only at the target location. Where the target is cancer cells, a number of targeting mechanisms exist to allow site-specific activation of carbohydrate inactivated prodrugs. Two broad classes of targeting mechanism envisioned by the invention for targeting to cancer cells are activating the prodrug by an enzyme that is over-produced in cancer cells relative to non-cancerous cells, and utilizing a cancer specific antibody to drive accumulation of a linked enzyme to the site of cancer cells, e.g., using antibody-directed enzyme targeting to activate a prodrug in a target tissue.

A monoclonal antibody reacting to a target antigen, linked to an enzyme, such as beta-galactosidase or alpha-galactosidase, will activate a prodrug to an active drug at those locations where the antibody accumulates. In one embodiment of the invention, a non-native enzyme is linked to a tumor specific antibody. The antibody-enzyme conjugate is administered to the patient such that the tumor specific antibody-enzyme conjugate accumulates in at the situs of the tumor. A conjugate-inactivated prodrug is administered to the patient. At the situs of the tumor, the level of the non-native enzyme is greater than in other tissues of the body, and the enzyme cleaves the conjugate from the conjugate inactivated prodrug, releasing the active drug. In this manner an insoluble or highly toxic drug is delivered to the tumor in a manner that leads to an accumulation of the active drug at the location where therapeutic benefit may be derived. Thus, the selective activation of the prodrug at a tumor site would result in improved anti-tumor activity with minimal side effects.

Linkage of an enzyme and antibody can be accomplished by a number of chemical means. The primary consideration is to fuse the antibody and the enzyme in a manner that neither interferes with the catalytic activity of the enzyme moiety nor interferes with the binding affinity of the antibody moiety for the target antigen. So long as the enzyme retains sufficient catalytic activity to activate the prodrug, and the antibody retains binding affinity to the target antigen, then the location of the conjugation between enzyme and antibody moieties will usually not limit the efficacy of the enzyme-antibody conjugate. See Kerr, D. E. et al., Bioconjugate Chem. 9: 255 (1998). It is not necessary that the catalytic portion of an enzyme-antibody conjugate be joined from two different molecules. Those skilled in the art also will recognize that it is possible to create recombinant fusion proteins that combine enzyme and antibody functions in a single molecule. Catalytic antibodies (abzymes) can be created by using recombinant DNA technology, allowing linkage and synthesis of an abzyme by fusion of the coding sequence of a target enzyme with the coding sequence of an antibody molecule, followed by expression of the fusion gene and translation of the abzyme protein structure.

A variety of antibodies have previously been identified that are useful for targeting enzymes to cancer cells. Such antibodies are discussed in the references cited in the background, and also include, but are not limited to HMFG1, H17E2, ING-1, mAb BR96, Anti-CEA, L6, 1F5, anti-MUC-1, and anti-sialamucin.

An example an antibody that is localized in neoplastic cells is monoclonal antibodies against the TAG-72 antigen (TAG-72 mAbs). Years of study indicate that TAG-72 mAbs may efficiently target tumor tissues. Radiolabeled anti-TAG mAbs have been utilized in radioimmunoguided surgery (RIGS) to provide surgeons with real-time information on disease extent and localization to enable precise resection. It has been shown that patients who have residual unresected RIGS-positive tissue remaining at the completion of surgery for primary colorectal cancer almost always succumb to their disease, even though traditional routine pathologic evaluation has failed to demonstrate residual tumor cells. Thus, in patients where complete resection is not possible due to the extent of the metastases or multiple lesions, by utilizing the TAG-72 mAbs to target site-specific activation of a chemotherapeutic drug with a antibody-directed enzyme complex allows directed chemotherapy against multiple metastases and potentially reduced mortality and morbidity from these cancers.

Clinical trials on the RIGS procedure demonstrate the efficacy of tumor targeting with the TAG-72 mAbs. The RIGS procedure provides the surgeon with biological information on disease extent via the detection of radiolabeled monoclonal antibodies specific to antigens associated with colorectal carcinoma using a hand-held gamma detecting probe in the operative field. Two clinical trials (T89-0023 and T90-0038) have been conducted with the RIGS procedure using the ¹²⁵I-radiolabeled murine mAbs B72.3 and CC49.

CTEP study T89-0023 evaluated the early anti-TAG-72 murine mAb, B72.3, radiolabeled with ¹²⁵I in 705 patients with primary and recurrent cancer. Patients were injected with 1 mg of B72.3 mAb labeled with 2 mCi of ¹²⁵I. The B72.3 mAb showed localization of visible colorectal tumors in 77% of patients with primary disease and in 72% of patients with recurrent disease. However, HAMA formation was reported in >50% of these patients. Results of these studies can be reviewed by referring to:
12) M. Cohen, E. W. Martin, Jr., I. Lavery, J. Daly, A. Sardi, D. Aitken, K. Bland, C. Mojzisik, and G. Hinkle. Radioimmunoguided surgery using iodine 125 B72.3 in patients with colorectal cancer. Arch. Surg. 126: 349-52 (1991).
13) A. Nieroda, C. Mojzisik, G. Hinkle, M. O. Thurston, and E. W. Martin, Jr. Radioimmunoguided surgery (RIGS) in recurrent colorectal cancer. Cancer Detect Prev. 15: 225-9 (1991).

CTEP study T90-0038 included both a dose ranging phase I evaluation of the ¹²⁵I-CC49 mAb and a phase 11 evaluation of intra-abdominal patterns of disease dissemination in 313 patients with primary and recurrent colorectal cancer¹⁰. This was the first clinical trial of the CC49 murine mAb recognizing TAG-72 antigen. Patients with primary and recurrent colorectal cancer were injected with 1 mg of CC49 mAb radiolabeled with 2 mCi of ¹²⁵I and later underwent standard surgical explorations using the RIGS probe. Resected tumors were evaluated histologically and were also analyzed for biologic expression of the TAG-72 antigen as detected by the RIGS system. Rates of localization using the CC49 mAb were 85% in patients with primary disease and 97% in patients with recurrent cancer.

A striking result of the T90-0038 study was the association of disease found in regions of the gastrohepatic ligament, celiac axis, and retropancreatic aorta with the development of liver metastases. In patients with primary colorectal carcinoma, 22% actually had visibly evident liver metastases, while 68% demonstrated RIGS positivity in the gastrohepatic ligament. In contrast, 64% of patients with recurrent colon cancer exhibited liver metastases, while 67% demonstrated RIGS positivity in the gastrohepatic ligament. Thus, RIGS-identified TAG-72 activity was demonstrated in the gastrohepatic ligament in nearly identical percentages of patients with primary and recurrent disease. Equally striking were identical RIGS findings in the celiac axis (49%) of both primary and recurrent patients. Finally, both patient populations showed similar distribution of tumor antigen in the retroperitoneum, and small bowel mesentery. The similarity of tumor antigen involvement in lymph node-bearing regions in both primary and recurrent patients strongly suggested that these areas are already involved in the disease process at the time of the first resection. Of note, there appeared to be no difference in pattern of tumor antigen spread based on primary tumor location. These findings imply that the disease process may be quite advanced in patients who present with what visibly appears to be disease of limited extent. These studies with the RIGS system demonstrate that the TAG-72 mAbs are associated with neoplastic lesions and when linked with an appropriate enzyme are expected to effectively target prodrug activation to the site of metastatic lesions arising from primary tumors bearing the TAG-72 antigen. Results of these studies can be reviewed by referring to:
14) M. W. Arnold, S. Schneebaum, A. Berens, L. Petty, C. Mojzisik, G. Hinkle, and E. W. Martin, Jr. Intraoperative detection of colorectal cancer with radioimmunoguided surgery and CC49, a second-generation monoclonal antibody. Ann. Surg. 216: 627-32 (1992).

The TAG-72 mAbs allow targeting of chemotherapeutic cytostatic agents to sites that would not typically be resected in traditional surgery. RIGS studies have shown a significant correlation between the presence of RIGS positive tissue and the presence of neoplastic cells. Martinez et al. conducted a comprehensive analysis of in RIGS in 212 patients with primary and recurrent colorectal cancer. To determine the prognostic significance of residual RIGS positive tissue at the completion of surgery, patients were divided into three groups; A) RIGS-negative (i.e., those with no evidence of residual RIGS-positive tissue); B) those with remaining RIGS-positive tissue, but no visible or gross tumor remaining; and C) those with RIGS-positive, gross tumor remaining. The 74 RIGS-negative patients had a significantly better survival at 5 years than did both of the RIGS positive groups. RIGS was able to clearly distinguish a subset of patients undergoing complete and traditionally “curative” resections that were doomed to recur and die of their disease at a rate similar to that of patients with incomplete resections. Thus, those patients that had remaining RIGS positive tissue (and therefore with tissue displaying the TAG-72 antigen) were likely to have unresected cancer cells. Effective targeting of cytostatic agents to those unresected cells is expected to improve treatment and long term prognosis of those patients.

Linkage of a label such as ¹²⁵I to an enzyme-antibody conjugate of the invention allows validation of the functionality of the accumulation of the conjugate preferentially in the target tissue. The RIGS probe can be employed as an adjunct to confirm that a targeting antibody used in an ADEPT procedure is actually functioning to target the activating enzyme. The labeled enzyme-antibody conjugate can be delivered to a patient, and the prodrug can be administered only after the activating enzyme has accumulated at the target tissue, and unbound enzyme-antibody conjugate has cleared from non-target tissues. Those skilled in the art will recognize that a number of different labels and detection methods are available that will allow monitoring of the accumulation of an enzyme-antibody conjugate in a target tissue.

To demonstrate the utility and enablement of the invention, we synthesized six carbohydrate-geldanamycin conjugates for possible delivery through ADEPT with beta-galactosidase or beta-glucosidase.

The structural study of GA-Hsp90 complexes demonstrates the invention of how conjugation of carbohydrate groups can be used to create prodrugs. While the mechanism of action of many drugs are not known with particularity, the inventive method for conjugating carbohydrates to active drugs, inactivating the drug, and creating a prodrug applies to many classes of drugs, as will be apparent to those skilled in the art. Moreover, for those drugs which are characterized as to structures critical for biological activity, skilled artisans will be able to readily identify functional groups which can be modified by the inventive method to create carbohydrate conjugated prodrugs.

Thus, the efficacy of the present invention is demonstrated by modification of GA by carbohydrate conjugates at the 7- or 11-position synthesis to create inactive prodrugs. Crystal structure of Hsp90-geldanamycin complex indicated that 85% of the surface area of GA is buried in the binding pocket present on the surface of Hsp90, while a benzoquinone group is positioned near the entrance of the pocket and this benzoquinone makes only a few contacts with the binding pocket of Hsp90. See Stebbins et al., Cell, 89: 239-250 (1997). By reviewing the crystal structure of the Hsp90-GA complex, some structural features can be deduced. Structure and activity relationship (SAR) studies revealed that the 7-position and 11-position of GA are critical for GA activity and modification of these two positions results in an inactive compound. The carbamate group in the 7-position is crucial in forming a hydrogen bond to Hsp90. Removal of the carbamate group or attachment of additional atoms changes binding affinity and abolishes GA activity. However, X-ray structure of geldanamycin shows that the free 11-OH group is buried inside the ansa ring. The limited space around 11-OH group and intramolecular H-bonding forces limit accessibility for direct glycosylation of the 11-OH group by bulky sugar donor. The low reactivity at the 7-position limits conjugation of other groups at the 7-position.

Crystal structure study suggests that an amino group substitution at C17 position of the benzoquinone may improve cellular activity indirectly by stabilizing the position of a quinone over the reduced hydroquinone. Previous results on GA derivatives revealed that modifications at the 17 position on the quinone ring maintained anticancer activity within the nanomolar range. Thus, conjugating a bulky sugar at the 17-position of GA will disrupt the stereochemistry of the binding interaction and affect the GA binding to Hsp90 thus making GA inactive against its chemical target.

One embodiment of the inventive method was to design and synthesize glucose-geldanamycin conjugates for site-specific activation by beta-glucosidase inside of tumor cells. The invention is embodied in glucose-GA conjugates that can be activated by beta-glucosidase glucosidase expressed inside of cancer cells. Cancer cells, due to their high demands for glucose relative to untransformed cells, express high levels of the beta-glucosidase enzyme relative to normal or untransformed cells. Therefore, a glucose-conjugated prodrug, such as glucose-GA, will be preferentially activated in cancer cells due to the high expression of beta-glucosidase. Those skilled in the art will recognize that by following the precepts of the present invention, many drugs can be inactivated by conjugation of glucose to the active drug to create a prodrug that can be activated by beta-glucosidase.

Glucose-GA showed anticancer activity with IC₅₀ of 70.2-380.9 nM (nanomolar) in various cancer cells through beta-glucosidase activation inside of the tumor cells. The specific activation of glucose-GA prodrug in cancer cells by native beta-glucosidase was demonstrated by the reduction of specific activity of the drug by the addition of the beta-glucosidase specific inhibitor 2,5-dihydroxymethyl-3,4-dihydroxypyrrolidine (DMDP) which completely abolished glucose-GA activity.

The present invention relieves the problems of low specificity and high toxicity along with low water solubility for GA. A series of carbohydrate-geldanamycin conjugates were synthesized, including glucose-GA, galactose-GA and lactose-GA. These conjugates were inactive prodrugs as expected. Conjugation was particularly carried out at the 17-position and resulting carbohydrate-GA conjugates were tested for bioactivity in a number of cancer cell lines in vitro. The enzyme-specific activation of these conjugates was evaluated with beta-glucosidase inhibitor and beta-galactosidase. Carbohydrate conjugation at the 17-position of GA converted the GA an inactive prodrug before enzyme cleavage.

In contrast to activation of glucose-GA conjugates by a native enzyme, galactose-GA conjugates, with a different chemical linkage than glucose-GA, will not be cleaved by beta-glucosidase, and remain inactive. Galactose conjugated prodrugs can, however, be activated by beta-galactosidase. Because beta-galactosidase is not expected to be enriched in cancer cells, prodrug activation can be targeted to cancer cells by linking beta-galactosidase to an antibody generated against antigens displayed by the target cancer. If galactose-prodrug conjugates are delivered to a target tissue in association with beta-galactosidase via ADEPT, the conjugates will be specifically activated by the enzymes at the tumor site and will exhibit targeted antitumor activity.

The galactose-GA conjugates, as expected, did not have any biological activity against human cells, even though the length of the linker is long enough that the bulky sugar moieties stretch out of the pocket when binding to Hsp90, since human tumor cells lack beta-galactosidase enzyme activity necessary to cleave the inactivating conjugate from the prodrug. However, galactose-GA conjugates incubated with exogenous beta-galactosidase are activated by cleavage of the galactose inactivating group. Thus, when galactose conjugated prodrugs are used in conjunction with beta-galactosidase delivered into the target sites by a specific antibody, a highly toxic drug can be activated at a restricted, specific location. Galactose-GA conjugated prodrugs can be used in the practice of ADEPT technology.

Compared to glucose-GA, galactose-GA and lactose-GA conjugates exhibited much less native activity with an IC₅₀ greater than 8000-25000 nM due to the absence of beta-galactosidase expression in the cancer cells. However, when galactose-GA and lactose-GA were incubated in the presence of beta-galactosidase with the cells, the specific activity of galactose-GA and lactose-GA was remarkably enhanced by 3 to 40-fold. The specific activity of the glucose-GA conjugates was not affected by a beta-galactosidase treatment.

The results indicate the specific substrates of beta-galactosidase and beta-glucosidase for activating carbohydrate-GA prodrugs. Glucose-GA can be selectively activated by native β-glucosidase, and activation is not affected by exogenous β-galactosidase. Galactose-GA and lactose-GA, on the other hand, are not substantially activated by native β-glucosidase and are substantially activated by exogenous 9-galactosidase. The general utility of the invention for reversibly inactivating a variety of drugs and creating prodrugs capable of targeted activation is demonstrated by the GA prodrugs of the invention. Skilled chemists will recognize that the creation of enzymatically accessible linkages of carbohydrate groups to drug molecules is broadly applicable for the creation of targetable prodrugs. Such prodrugs are useful in a variety of applications, including for ADEPT.

Therefore, carbohydrate-GA conjugated prodrugs are expected to be a targeted substrate in drug therapy for cancer. Glucose-GA prodrug activated by beta-glucosidase overexpressed in neoplastic cells represents only one of many different carbohydrate-drug prodrug conjugates that can be activated by antibody-directed enzyme prodrug therapy (ADEPT). Galactose-GA prodrug can be activated in a site-specific manner by linking beta-galactosidase to a tumor specific antibody for site-specific activation in tumors, thus minimizing the toxicity of GA. Similarly, alpha-galactosidase or other activating enzymes can be linked to tumor-specific antibodies to target activating enzymes to a target tissue. The particular enzyme chosen for linkage to a tumor-specific antibody will be dependent on the nature of the linkage of the inactivating group to the drug compound moiety of the conjugated prodrug.

Another embodiment of the inventive method was to design and synthesize galactose-GA conjugate with galactose attached to GA at C17 position through an amine linker with varied lengths. Thus, this embodiment of the carbohydrate conjugated prodrug is inactivated by the addition of galactose; but, when the inactivating carbohydrate group is cleaved, the amine linker remains, increasing biological activity.

Anticancer Activity of GA Prodrug Conjugants

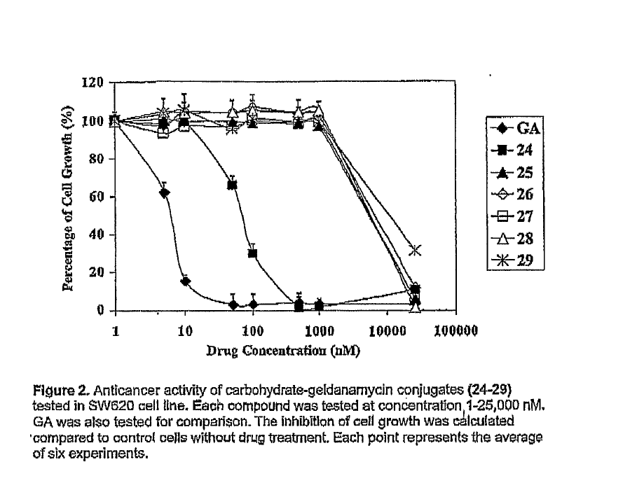

The anticancer activities of six prodrug conjugates synthesized by the method were tested for anticytostatic activity by using the MTS assay to show suppression of cell growth. Four different human cell lines, including colorectal carcinoma cells (SW620, HT 29), breast cancer cells (MCF7), and leukemia cells (K562), were tested using the MTS assay. The results are shown in FIGS. 2-4. Each compound was tested at concentration 1-25,000 nM. GA also was tested for comparison. The inhibition of cell growth was calculated compared to control cells without drug treatment. For purposes of this description of in vitro cell culture testing, drug concentrations are given for the molar concentrations of the chemotherapeutic agents present in the media at the initiation of the test. Skilled artisans are aware of the various conversion factors necessary to convert media concentrations to tissue concentration targets and the dose necessary to achieve such a dose.

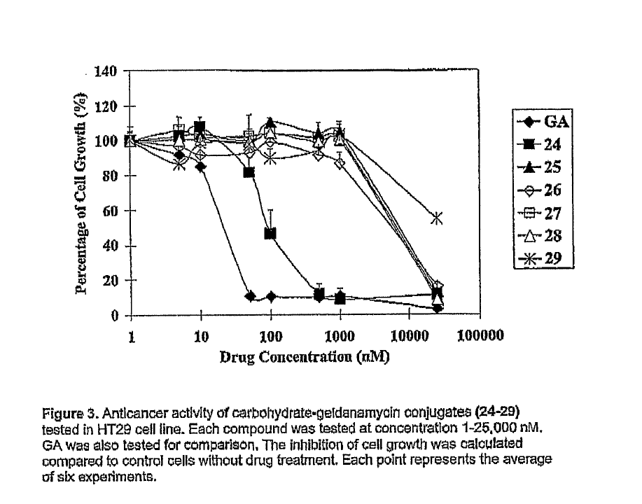

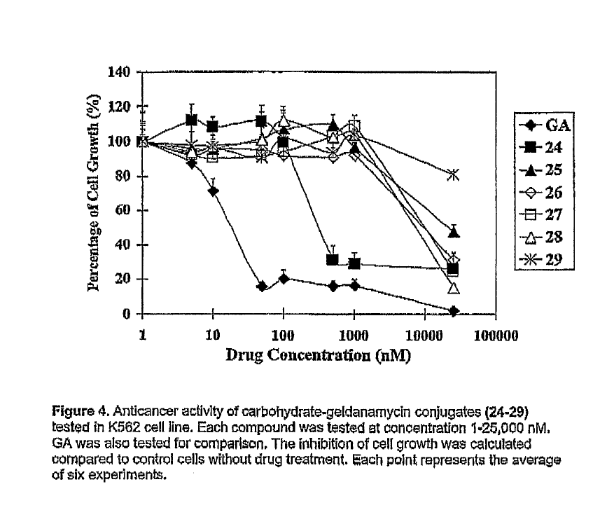

FIG. 2 shows a graph showing relative growth of SW620 cells in the presence of carbohydrate-geldanamycin conjugates (24-29) as a function of drug concentration in nanomoles/liter versus relative percentage of cell growth. FIG. 3 shows a graph showing relative growth of HT29 cells in the presence of carbohydrate-geldanamycin conjugates (24-29) as a function of drug concentration in nanomoles/liter versus relative percentage of cell growth. FIG. 4 shows a graph showing relative growth of K562 cells in the presence of carbohydrate-geldanamycin conjugates (24-29) as a function of drug concentration in nanomoles/liter (nM) versus relative percentage of cell growth. Each point represents the average of six experiments.

As expected, only the glucose-geldanamycin (compound 24) showed anticytostatic activity against these tumor cells, while all other four galactose-geldanamycin conjugates with different linker and lactose-GA were relatively inactive. For example, in SW620 cell line, compound 24 showed anticancer activity with an IC₅₀ of 70 nM, while the other conjugates 25, 29 with the same spacer, but different sugar moieties, showed low activity with IC₅₀>800 nM, and 20000 nM, respectively.

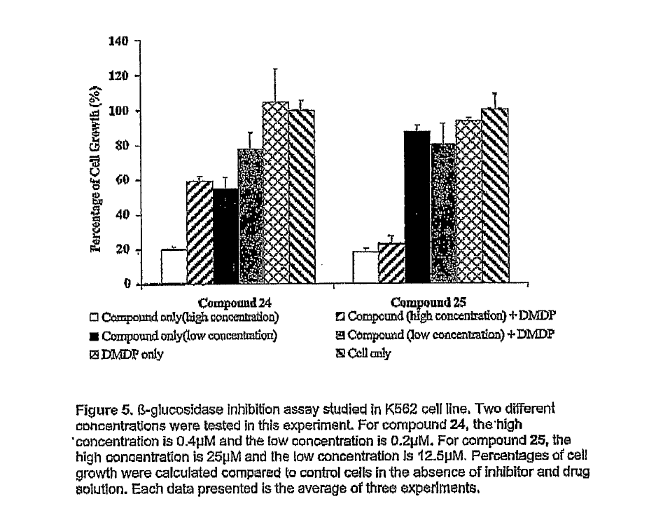

Similar results showing preferential activation of glucose conjugates relative to galactose or lactose conjugates were obtained in other cancer cell lines. The glucose-conjugated compound is apparently cleaved by native glucosidase inside of the cancer cells resulting in release of a biologically active anti-cytostatic compound. The cleavage of glucose conjugates by an enzymatic pathway to release an active drug is shown by a beta-glucosidase inhibition assay. FIG. 5 is a graph showing the results of a beta-glucosidase inhibition assay studied in K562 cell line. Two different concentrations of prodrug were tested in this experiment. For compound 24, 17-demethoxy-17-[(2-β-glucopyranosylethyl)amino]geldanamycin], the higher concentration is 0.4 μM and the lower concentration is 0.2 μM. For compound 25, 17-demethoxy-17-[(2-β-galactopyranosylethyl)amino]geldanamycin], the higher concentration is 25 μM and the low concentration is 12.5 μM. Percentages of cell growth were calculated compared to control cells in the absence of beta-glucosidase inhibitor and prodrug solution. Each bar represents the average of three experiments. These results demonstrate that the cytostatic effects of the glucose conjugate compound 24 are limited by inhibitors of beta-glucosidase, while the cytostatic effects of galactose conjugates is relatively unaffected by the addition of the beta-glucosidase inhibitor DMDP. While the higher concentration of compound 24 limits cells growth to about 20% of untreated cells, when the beta-glucosidase inhibitor DMDP is added, the high concentration of compound 24 only reduces growth to about 60% that of untreated cells. The galactose conjugate, compound 25 is relatively unaffected by the addition of DMDP.

In contrast, when beta-galactosidase is presented to compounds 25-29, possessing various linkages of galactose or lactose to geldanamycin, the cytostatic activity of the drug is dramatically increased, while the relative ability of glucose conjugate compound 24 is relatively unaffected.

Table 2 summarizes the IC₅₀ of all six geldanamycin-sugar conjugates including GA.

TABLE 2
Anticancer activity (IC₅₀) of carbohydrate-geldanamycin
in four different cancer cell lines (nM)

| | GA | 24 | 25 | 26 | 27 | 28 | 29 |
|---|---|---|---|---|---|---|---|
| SW620 | 6.2 | 70.2 | >8000 | >14000 | >13000 | >11000 | >20000 |
| HT29 | 24.5 | 104.7 | >14000 | >6000 | >13000 | >13000 | >25000 |
| MCF7 | 6.5 | 754.0 | >25000 | >12000 | >17000 | >22000 | >22000 |
| K562 | 22.1 | 380.9 | >23000 | >14000 | >19000 | >15000 | >25000 |

Beta-Galactosidase Cleavage Assay

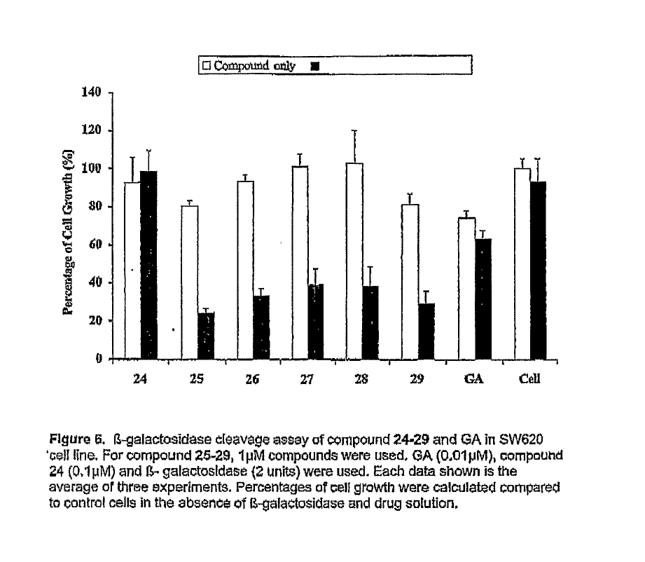

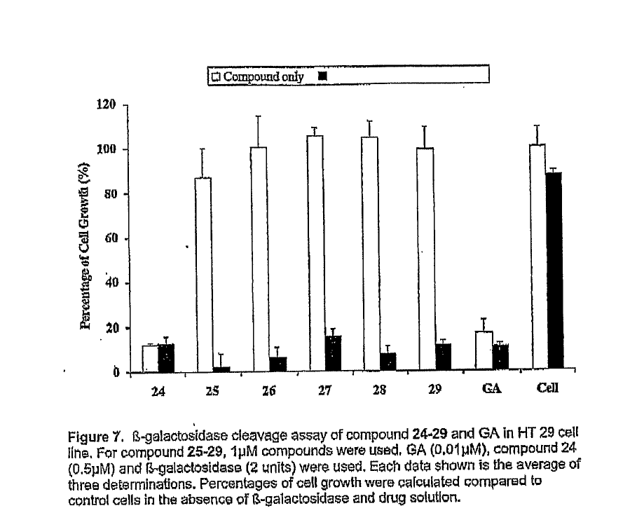

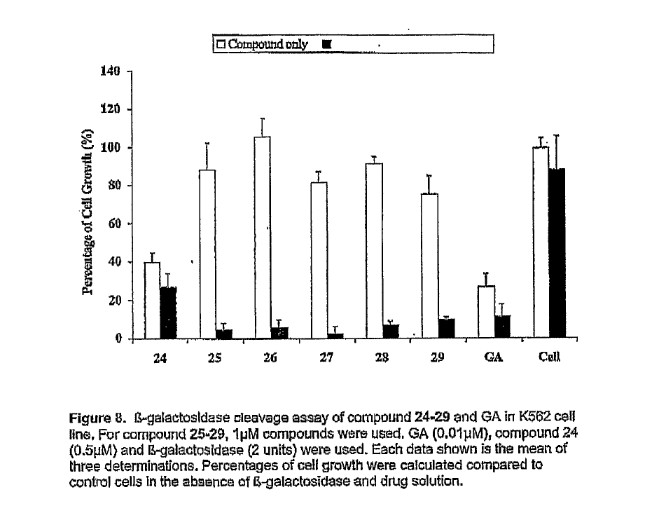

Since beta-glucosidase failed to activate compound 25, the facility of beta-galactosidase to activate galactoside-linked prodrugs was subsequently evaluated. Thus, galactose-GA and lactose-GA conjugates were exposed to beta-galactosidase and observed for enhancement of anticancer activities. FIG. 6 shows a graph showing results of a beta-galactosidase cleavage assay of carbohydrate-geldanamycin conjugates (24-29) and GA in the SW620 cell line as a function of treatment composition versus relative percentage of cell growth. For compounds 25-29, 1 μM concentration of prodrug compounds were used. GA (0.01 μM), compound 24 (0.1 μM) and beta-galactosidase (2 units) was used in the cell culture. Each bar in the graphs shown is the average of three experiments. Relative percentage of cell growth was calculated compared to control cells in the absence of beta-galactosidase and drug solution. Cell lines SW620, HT29 and K562 were exposed to 2 units of beta-galactosidase and then treated individually with 1 μM of compounds 25-29. At this concentration, galactose-geldanamycin conjugates alone were relatively devoid of anticancer activity. However, all five inactive galactose GA conjugates and the lactose-GA conjugate were activated with 3 to 40-fold increase in their activity against HT29 and K562 after incubation with beta-galactosidase (See FIGS. 6-8). Beta-galactosidase treatment of the glucose-GA conjugate 24 and GA did not affect biological activity.

These results demonstrate that the bulky sugar moiety at C-17 position can abolish the anticancer activity of geldanamycin, and the inactive carbohydrate prodrugs of geldanamycin can be site-specifically activated by beta-glucosidase and beta-galactosidase. Interestingly, compounds 25-29 showed nearly the same activity as GA after being cleaved by 11-galactosidase. This indicates that the length of spacer has little effect on anticancer activity. Although lactose is a disaccharide, its structure is galactose β-1,4 glucose. β-galactosidase could cleave the terminal galactose in the lactose structure leaving the glucose moiety, which can be subsequently cleaved by the R-glucosidase in the cells. Since these compounds respond to the presence of an exogenous enzyme they demonstrate their utility for exploitation of targeted drug release via ADEPT.

Those skilled in the art of carbohydrate chemistry will recognize that conjugation of complex carbohydrates to active drug compounds will be likely to inactivate the conjugated drug. Attachment of a carbohydrate of 5 carbons or more is likely to inactivate a drug. If the linkage of the carbohydrate is created with a linkage cleaved by an enzyme of biologic origin, then the prodrug is expected to be activated when in the presence of an enzyme capable of cleaving that linkage. In the present invention new carbohydrate-geldanamycin conjugates were synthesized through C-17 linkage of geldanamycin. The carbohydrate conjugation of geldanamycin through the C-17 position produces inactive prodrugs. The glucose-GA conjugate showed high anticancer activity with IC₅₀ within the nanomolar range, while galactose-geldanamycin conjugates showed much less activity. These prodrugs can be activated by enzymatic cleavage. The glucose-geldanamycin prodrugs can be site-specifically activated by beta-glucosidase enriched in tumor cells compared to other cells derived from the same tissue. The galactose-geldanamycin prodrugs can be site-specifically activated by beta-galactosidase showing 3 to 40-fold increase in anticancer activity in the presence of beta-galactosidase compared to the galactose-geldanamycin alone. This is achieved through the cleavage of galactose moiety by beta-galactosidase. These carbohydrate-geldanamycin conjugates can be used for antibody-directed enzyme prodrug therapy (ADEPT) by linkage of the activating enzyme to an antibody which target antigen is enriched in the targeted cells.

The results also indicate that the length of the spacer, between carbohydrate and geldanamycin in the conjugates, does not seem to play a significant role in the anticancer activity after enzyme cleavage. The linker at C-17 position remained attached to geldanamycin even after the enzymatic cleavage of the sugar moiety, yet substantial cytostatic activity remained. Further variations and optimization of the linker structure to modulate activity and geldanamycin prodrugs with a labile at the C-11 position attaching the modifying sugar are contemplated by the invention.

These results have demonstrated that C-17 position modification of geldanamycin with sugar moieties diminishes its anticancer activity. However, these conjugated prodrugs still exhibit residual activity at high concentrations, while the enzyme-cleaved geldanamycin-sugar prodrugs with an extra linker segment at C-17 position exhibits increased activity. Although direct glycosylation of GA at the C-11 position is difficult, the modification through a labile linker is feasible to completely abolish its anticancer activity before enzyme cleavage. Previous work has shown that a short carbamate linkage between the sugar residue and the bulky aglycone dramatically decreases the kinetics of hydrolysis of the glycosidic bond. Several structurally different enzymes have been shown to cleave anticancer prodrugs containing a self-elimination spacer much more readily than the corresponding prodrugs without a spacer. This could be due to the highly sterically hindered environment that makes enzyme attack on the glycoside linkage very difficult without a labile linker. In some cases, the enzymatic release of the active drug from the conjugated prodrug complex without a spacer did not occur at all. This clearly demonstrates the beneficial effects of incorporation of a spacer on prodrug activation characteristics. In addition, the rapid non-specific prodrug hydrolysis was observed in plasma if drug-spacer had an ester linkage. An o-nitro-, instead of p-nitro-, substituted phenol linker can induce rapid spacer elimination.

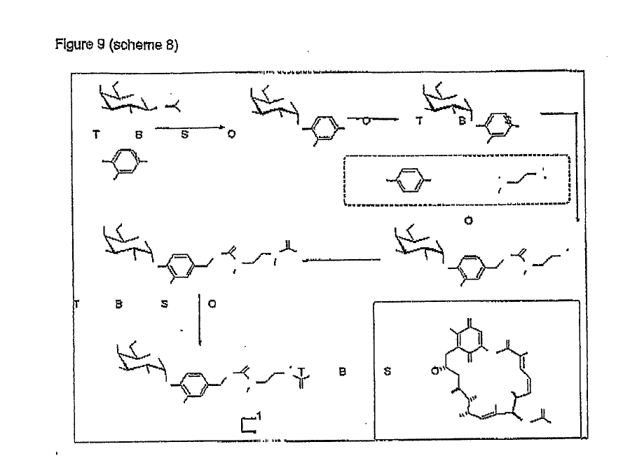

In light of all these previous results the invention is also embodied in the use of a self-elimination spacer designed based on electronic cascade chemistry. Condensation of fully silylated isopropyl β-D-galactopyranoside (IPTG) with 3-nitro-4-hydroxybenzaldehyde will give α-glycoside 2 as a major product with NIS and AgOTf as catalysts. Reduction of aldehyde group of 2 with NaBH₄ in MeOH will afford 3 containing a benzyl alcohol group. This hydroxyl will be activated as a p-nitrophenylchlorocarbonate, followed by condensation with mono protected diamine in one-pot affording 4. See FIG. 9 (scheme 8). Dry 3 M HCl in EtOAc will remove the Boc protecting group from 4 giving the deprotected amine as a hydrochloride salt. Without purification, this salt will be used directly to react with phosgene leading to the activated carbamoyl chloride 5. The coupling reaction between 5 and geldanamycin will be achieved in TEA with the aid of DMAP. The four silyl groups of the resulting compound 6 will be removed with an excess of HF-pridine complex in dry acetonitrile. With an elongated spacer, it is expected that the prodrug will be efficiently activated by α-galactosidase under physiological condition. Obviously, we are able to prepare α-glycoside having 3-nitro-4-hydroxybenzyl alcohol spacer as a prodrug back-up.

Thus, after cleavage of the sugar moiety, and elimination of a labile linker structure, the original geldanamycin will be released in the tumor site without any extra linker structure.

The following Examples show how the present invention has been practiced, but should not be construed as limiting. In this application, all citations are expressly incorporated herein by reference.

### EXAMPLE 1

In practicing the invention, in addition to those techniques familiar to those skilled in the art of chemistry, molecular biology, cell biology, and immunology, the following specific methods were practiced:

Beta-glucosidase Inhibition assay. To verify if the cleavage of sugar moieties from compound 24 is crucial for the anticancer activity, we performed the β-glucosidase inhibition study in K562 cell line. The K562 cells were treated with β-glucosidase inhibitor 2, 5-dihydroxymethyl-3,4-dihydroxypyrrolidine (DMDP) solution (100 μM) for 10 minutes before adding carbohydrate-geldanamycin conjugates. The concentrations of glucose-GA were 0.4 μM and 0.2 μM, respectively. Indeed, after treatment with DMDP, the activity of compound 24 decreased significantly by 3-fold, while DMDP did not affect the activity of compound 25 (FIG. 4). DMDP alone did not have any effect on cell growth. From the structures of compounds 24 and 25, the only difference between these two compounds is the sugar moiety, which is glucose in compound 24 and galactose in compound 25. The results suggest that the anticancer activity of compound 24 is achieved through cleavage of glucose moiety by beta-glucosidase in the cells, while low activity of compound 25 is due to the lack of beta-galactosidase in the cells for the cleavage of galactose moiety.

Cell Culture. Cell lines SW620, HT29, MCF7, and K562 were cultured in RPMI 1640 (Invitrogen, Carlsbad, Calif., USA) supplemented with 10% fetal bovine serum (FBS), 1% non-essential amino acid and Penicillin (100 u/mL) Streptomycin (100 ug/mL) in a humidified atmosphere of 5% CO₂ and 95% air at 37° C. The culture mediums were changed every 2-3 days.

Anticancer activity of compound (24-29) (MTS assay). Cells (2,000-10,000) were seeded in 96 well plates in RPMI-1640 and incubated for 24 hours. The exponentially growing cancer cells were incubated with various concentrations of compounds for 72 hours at 37° C. (5% CO₂, 95% humidity). After 72 hours incubation, tetrazolium [3-(4,5-dimethylhiazol-2-yl)]-5-(3-carboxymethoxyphenyl)-2-(4-sulfophenyl)-2H-tetrazolium, inner salt (MTS, 2 mg/ml) and phenazine methosulfate (PMS, 25 μM were mixed and added directly to the cells. After incubation for 3 hrs at 37° C., the absorbance of formazan (the metabolite of MTS by viable cells) was measured at 490 nm. The IC₅₀ values of the carbohydrate-drug conjugates for anti-proliferation were calculated by the dose-response curves of percentage of cell growth vs. control (no compound added).

Production of geldanamycin. Geldanamycin was isolated from the fermentation broth of Streptomyces hygroscopicus NRRL3602. Fermentation and chemical isolation was carried out per method of DeBoer et al. J. Antibiotics, 23: 442-447 (1970).

Analytical criteria. The NMR data were recorded on a Varian-400 or 500 MHz spectrometer. MS spectra were obtained from a Kratos MS 80 spectrometer using electrospray ionization mode (ESI) or electronic ionization (E1). The MTS assay was measured on a DYNEX MRX microplate reader. Silica gel F254 plates (Merck) and Silica Gel 60 (70-230 mesh) were used in analytical thin-layer chromatography (TLC) and column chromatography, respectively.

Beta-Glucosidase inhibition assay for compound 2425. K562 Cells (2,000 cells/80 μL RPMI-1640) were seeded in 96 well plates and incubated for 24 hours. The exponentially growing cancer cells were incubated in three different conditions (cells with different concentration of test compounds and DMDP, cells with different concentration of test compounds only, and cell with DMDP only) for 72 hours at 37° C. (5% CO₂, 100% humidity). The control cells were exposed to fresh medium only. Then, the anticancer activities of compound 24, 25 were measured with MTS assay as described above. The percentages of cell growth for tested compounds vs. control cells were calculated by the absorbance at 490 nm.

Beta-galactosidase cleavage assay. Cells (2,000-10,000) were seeded in 96 well plates in RPMI-1640 and incubated for 24 hours. The exponentially growing cancer cells were incubated with 2 units of beta-galactosidase for 10 minutes. The same concentration solutions of various compounds (25-29) were added to each well. The control cells were exposed to fresh medium only or to beta-galactosidase alone. After incubated for 72 hours at 37° C. (5% CO₂, 100% humidity), the cell growth was measured with MTS assay as described above. The percentages of cell growth of tested compounds vs. control were calculated by measuring the absorbance at 490 nm.

### EXAMPLE 2

The following examples demonstrate procedures employed in the practice of the invention, namely the chemical syntheses and purifications to produce carbohydrate conjugated prodrugs or other useful compounds.

Chemical Synthesis Strategy. The generalized strategy for synthesizing geldanamycin-sugar prodrugs is as follows. The trichloroacetimidate derivatives of sugar are first prepared as glycosyl donors from corresponding per-acetylated sugars in two steps with high yield. Glycosylation of these glycosyl donors with different precursors of aminoalcohols provides a series of protected sugar-primary amine derivatives. Next, deprotection and/or reduction furnishes the synthesis of free sugar-amine derivatives which can be linked to GA.

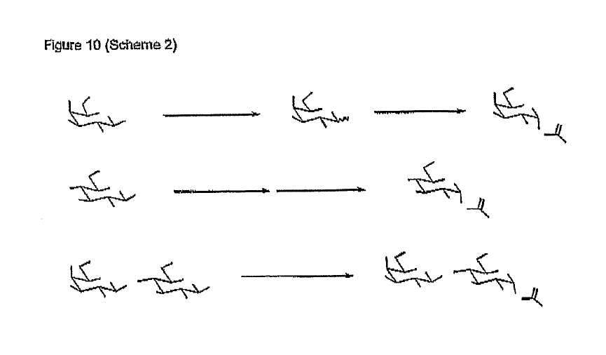

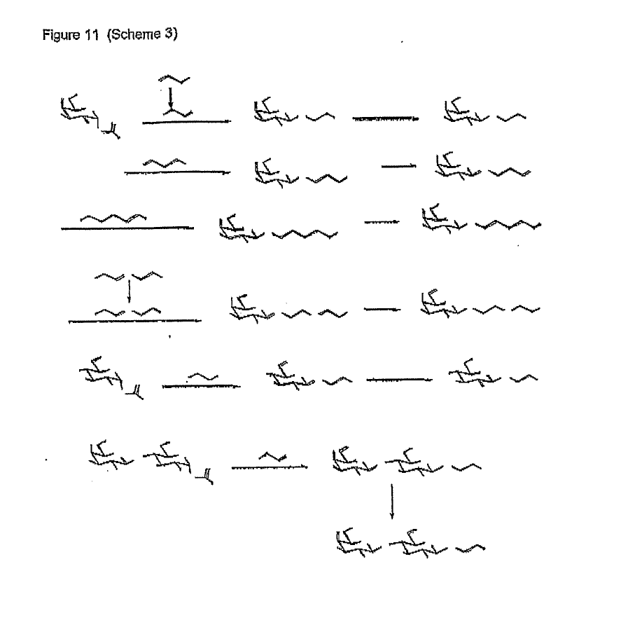

Using this method, six different geldanamycin-sugar prodrugs were synthesized. As shown in FIG. 10, Scheme 2, three trichloroacetimidate derivatives (compound numbers 3, 5, 7) of sugar (galactose, glucose and lactose) were prepared as glycosyl donors from corresponding per-acetylated sugars in two steps with high yield. Selective removal of the acetyl group at the anomeric position was achieved under a mild basic condition. Treatment of the free sugar with trichloroacetonitrile in presence of DBU to afford the anomerically pure, stable α-trichloroacetimidates. As shown in FIG. 11, Scheme 3, treatment of α-trichloroacetimidates (3, 5, 7) with different alcohol-linkers (8, 9, 10, 11) in the presence of trimethylsilyl trifluoromethanesulfonate (TMSOTf) afforded a series of protected sugar-primary amine derivatives (12-15, 20, 22) in beta-configuration. The free sugar-primary amine derivatives (16, 17, 18, 19, 21, 23) were obtained in high yields by two steps of deprotection

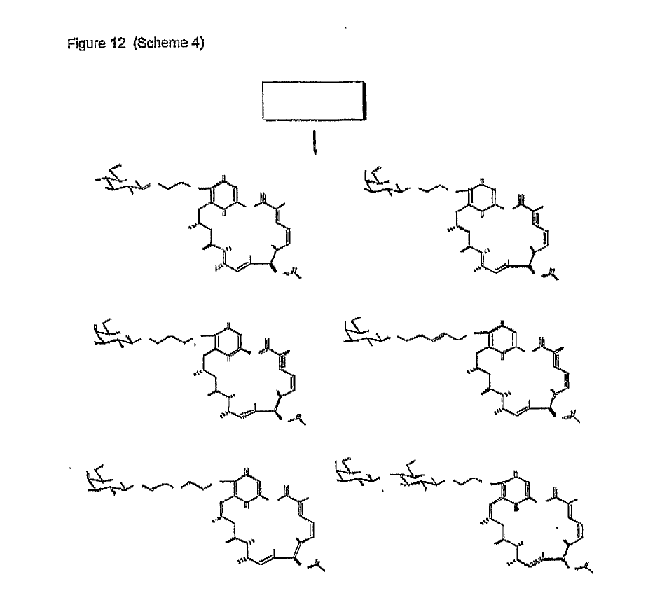

Coupling sugar-amine derivatives (21, 16, 17, 18, 19, 23) with GA in DMF (dimethyl formamide) at room temperature to produce type 11 sugar-geldanamycin conjugates in yield of 70-90%. GA was readily losing its 17-methoxy group in such mild conditions and forming 17-amino-17-demethoxygeldanamycin derivatives. As shown in FIG. 12, Scheme 4, six conjugates (24-29) with different sugar moieties or linkers were prepared.

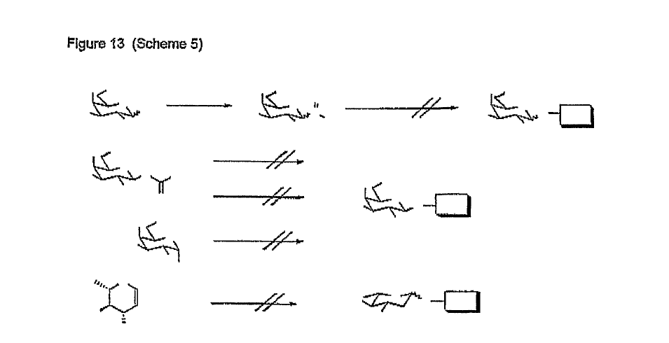

A series of methods were used to direct glycosylation of geldanamycin at the C11 position. The results, however, were not as efficient as conjugation at C17. As shown in FIG. 13, Scheme 5, only some of these glycosylation conditions produced desired results. Two reasons might account for the lack of uniform conjugation: 1) the solubility of GA was low in organic solvents that were used in glycosylation (such as methylene chloride and toluene); and 2) as evidenced by the X-Ray structure of geldanamycin, the C11 OH group is buried inside the ansa ring. The limited space around C11-OH group limits accessibility of direct glycosylation with bulky and rigid glycosyl donors. Intramolecular H-bonding forces may also exist in this molecule, thus making this —OH group less active. Smaller and more flexible carbohydrate donors with linkers are expected to have greater efficiency of conjugation at the C11 position.

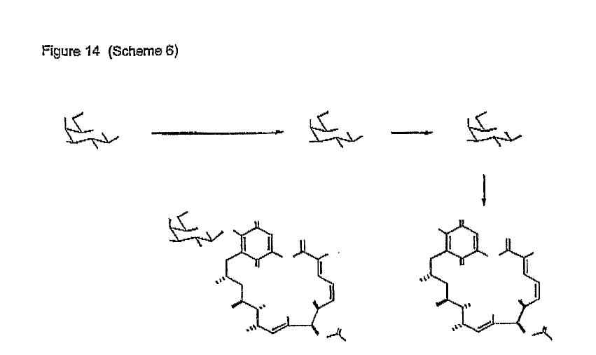

A direct linkage of glycosylamine (32) to GA was an inefficient reaction. Forty percent (40%) of GA starting material was recovered even after 3 days. The major product obtained was 17-amine-17-demethoxy-GA (34). (FIG. 14, Scheme 6).

General synthetic procedure of α-glycopyranosyl trichloroacetimidate:

Hydrazine acetate (0.89 g, 9.6 mmol) was added to a solution of 1, 2, 3, 4, 6-penta-O-acetyl-pyranose (8.1 mmol) in DMF (10 mL) at 60° C. under Ar. When thin layer chromatography (TLC) (solvent 1:1 hexanes-EtOAc) showed the formation of a new product and the disappearance of starting material, the mixture was diluted with EtOAc, washed with aqueous 5% NaCl (2×) and water, dried over Na₂SO₄, and precipitated with toluene to give the crude product. A solution of this crude product then was reacted with trichloroacetonitrile (10 eq), and DBU (2 eq) in CH₂Cl₂ (30 mL) for 60 min at 0° C. When TLC (2:1 hexane-EtOAc) showed the conversion to be complete, the solution was concentrated and purified by flash chromatography to yield the product.

The chemical properties of the synthesized α-glycopyranosyl trichloroacetimidates were as follows:

2,3,4,6-tetra-O-acetyl-α-galactopyranosyl trichloroacetimidate (3): ¹H NMR (CDCl₃): δ 8.67 (s, 1H), 6.61 (d, 1H, J=3.4 Hz), 5.56, (dd, 1H, J=3.0, 1.4 Hz), 5.43 (dd, 1H, J=10.8, 3.0 Hz), 5.36 (dd, 1H, 10.8, 3.4 Hz), 4.44 (m, 1H), 4.17 (dd, 1H, J=6.6, 11.3 Hz), 4.08 (dd, 1H, 6.7, 11.3 Hz), 2.17, 2.03, 2.02, 2.01 (4 s, each 3H). ¹³C NMR (CDCl₃): δ 170.35-169.31 (4COCH₃), 160.82, 93.45, 68.85, 67.38, 67.32, 66.47, 61.10, 20.11-19.85 (4 COCH₃). MS ES⁺ m/z: 514.17 (M+Na).

2,3,4,6-tetra-O-acetyl-α-glucopyranosyl trichloroacetimidate (5): ¹H NMR (CDCl₃) δ 8.70 (s, 1H), 6.57 (d, 1H, J=3.6 Hz), 5.14 (dd, 1H, J=3.7, 10.2 Hz), 2.08, 2.05, 2.04, 2.02 (4 s, each 3H). ¹³C NMR (CDCl₃): δ 169.95-169.02 (4COCH₃), 160.11, 92.55, 90.32, 69.65, 69.40, 69.31, 67.45, 61.08, 20.23-20.01 (4COCH₃). MS ES⁺ m/z 514.13 (M+Na).

2,3,4,6-tetra-O-acetyl-α-lactopyranosyl trichloroacetimidate (7): ¹H NMR (CDCl₃): δ 8.65 (s, 1H), 6.48 (d, 1H, J=3.6 Hz), 5.55 (t, 1H, J=9.9 Hz), 5.35 (d, 1H, J=3.0 Hz), 5.15-5.03 (m, 2H), 4.97-4.92 (dd, 1H, J=3.6, 10.2 Hz), 4.52-4.46 (m, 2H), 4.18-4.05 (m, 4H), 3.89-3.83 (m, 2H), 2.15 (s, 3H), 2.10 (s, 3H), 2.06 (s, 6H), 2.04 (s, 3H), 2.00 (s, 3H), 1.96 (s, 3H). ¹³C NMR (CDCl₃): δ 170.31-169.28 (7COCH₃), 161.21, 101.47, 93.10, 76.15, 71.36, 71.13, 70.93, 70.18, 69.78, 69.33, 66.79, 61.72, 60.98, 21.11-20.74 (7COCH₃). MS ES⁺ m/z: 802.19 (M+Na).

Synthesis of 2-Azidoethanol (8): 2-chloroethanol (25.2 mL, 375 mmol) was added to a solution of NaN₃ (30 g, 461 mmol) and NaOH (1.5 g, 37.5 mmol) in water (115 mL). The mixture was stirred at room temperature for 3 days, and sodium sulfate (35 g) was added. After 10 min, the mixture was extracted with CH₂Cl₂ (3×70 mL). The combined extracts were dried in Na₂SO₄ and concentrated. The residue was distilled to give 2-azidoethanol. ¹H NMR (CDCl₃): δ 3.79-3.76 (t, 2H, J=5.1 Hz), 3.47-3.44 (t, 2H, J=5.1 Hz), 2.02 (s, 1H). ¹³C NMR (CDCl₃): δ 61.76, 53.75. MS ES⁺ m/z: 88.12 (M+H).

Synthesis of 2-(2-azidoethoxy)-ethanol (9): NaN₃ (4.5 g, 69 mmol), tetrabutylammonium iodide (2.5 g, 6 mmol) and 18-crown-6 (10 mg) were added to a solution of 2-(2-chloroethoxy) ethanol (5 mL, 47 mmol) in 2-butanone (25 mL). The mixture was refluxed at 90° C. for 2 days. When ¹³C NMR spectroscopy of the supernatant showed the absence of a signal at δ 42.7 and the presence of a strong signal at δ 50.9 ppm, the mixture was filtered. The precipitate was rinsed with acetone and the combined solutions were concentrated. Distillation of the residue gave the pure product. ¹H NMR (CDCl₃): δ 3.74-3.71 (t, 2H, J=4.5 Hz), 3.68-3.65 (t, 2H, J=5.1 Hz), 3.59-3.56 (t, 2H, J=4.2 Hz), 3.40-3.37 (t, 2H, J=5.4 Hz), 2.41 (s, 1H). ¹³C NMR (CDCl₃): δ 72.65, 70.25, 61.94, 50.92. MS ES⁺ m/z: 132.13 (M+H).

General glycosylation procedure of O-acetyl-pyranosyl trichloroacetimidate: The trichloroacetimidate (1.63 mmol) was dissolved in dry CH₂Cl₂ (10 ml) with glycosyl acceptor (the primary alcohols, 1.5 eq) and 4 A MS (100 mg). The mixture was stirred at room temperature for 1 hr. After cooled to −30° C., TMSOTf (1 eq) was added. The reaction was stirred at −30° C. for 1 hr and then at room temperature over night. The reaction was quenched with saturated NaHCO₃. The mixture was extracted with CH₂Cl₂ and washed with NaHCO₃ and brine. The organic phase was dried over Na₂SO₄, filtered, and concentrated under vacuum. The residue was purified with chromatography (silica gel, hexane/EtOAc 7:3-1:1) to give the product.

The chemical properties of the synthesized O-acetyl-pyranosyl trichloroacetimidate are as follows:

2-azidoethyl 2,3,4,6-tetra-O-acetyl-b-galactopyranoside (12): ¹H NMR (500 MHz, CDCl₃): δ 5.40 (d, 1H, J=3.5 Hz), 5.26-5.23 (dd, 1H, J=8.0, 10.5 Hz), 5.04-5.01 (dd, 1H, J=10.5, 3.5 Hz), 4.56 (d, 1H, J=8.0 Hz), 4.21-4.11 (m, 2H), 4.07-4.03 (dt, 1H, J=10.0, 4.5 Hz), 3.93-3.91 (t, 1H, J=8.0 Hz), 3.72-3.67 (m, 1H), 3.54-3.49 (m, 1H), 3.32-3.28 (dt, 1H, J=13.5, 3.5 Hz), 2.16, 2.07, 2.05, 1.99 (4 s, each 3H). ¹³C NMR (CDCl₃): δ 170.41-169.68 (4 COCH₃), 101.38, 71.12, 71.03, 68.74, 68.65, 67.21, 61.47, 50.79, 21.03-20.83 (4 COCH₃). MS ES⁺ m/z: 440.23 (M+Na).

3-Cbz-amino-1-propyl-2,3,4,6-tetra-O-acetyl-β-galactopyranoside (13): ¹H NMR (400 MHz, CDCl₃): δ 7.35-7.30 (m, 5H), 5.36 (d, 1H, J=3.6 Hz), 5.21-5.15 (dd, 1H, J=10.2, 7.8 Hz), 5.07 (br, 3H), 5.01-4.96 (dd, 1H, J=10.5, 3.9 Hz), 4.44 (d, 1H, J=8.1 Hz), 4.14-4.11 (dd, 2H, J=6.6, 3.0 Hz), 3.95-3.85 (m, 2H), 3.66-3.53 (m, 1H), 3.36-3.18 (m, 2H), 2.11, 2.02, 2.01, 1.96 (4 s, each 3H), 1.80-1.75 (m, 2H). ¹³C NMR (CDCl₃) δ 170.65, 170.49, 170.36, 169.80, 156.67, 136.87, 128.72, 128.33, 128.29, 101.39, 71.05, 70.93, 68.97, 67.88, 67.24, 67.02, 66.78, 61.54, 38.45, 29.75, 20.94, 20.87, 80.80. MS ES⁺ m/z: 562.16 (M+Na).

5-Cbz-amino-1-pentyl-2,3,4,6-tetra-O-acetyl-β-galactopyranoside (14): ¹H NMR (400 MHz, CDCl₃) δ 7.33-7.30 (m, 5H), 5.35 (d, 1H, J=3.6 Hz), 5.19-5.13 (dd, 1H, J=−10.2, 7.8 Hz), 5.0 (s, 2H), 5.01-4.96 (dd, 1H, J=10.5, 3.6 Hz), 4.90 (br, 1H), 4.42 (d, 1H, J=7.8 Hz), 4.18-4.06 (m, 2H), 3.88-3.82 (m, 2H), 3.48-3.40 (m, 1H), 3.17-3.11 (m, 2H), 2.10, 2.01, 2.00, 1.95 (4 s, each 3H), 1.59-1.43 (m, 4H), 1.37-1.30 (m, 2H). ¹³C NMR (CDCl₃): δ 170.62, 170.51, 170.38, 169.66, 156.64, 136.87, 128.70, 128.28, 101.48, 71.12, 70.79, 70.06, 69.13, 67.28, 66.75, 62.73, 61.49, 41.08, 32.43, 29.78, 29.17, 23.23, 23.08, 20.94, 20.86, 20.79. MS ES⁺ m/z: 590.20 (M+Na).

5-azido-3-oxapentyl-2,3,4,6-tetra-O-acetyl-β-galactopyranoside (15): ¹H NMR (400 MHz, CDCl₃): δ 5.35 (d, 1H, J=3.2 Hz), 5.20-5.16 (dd, 1H, J=10.4, 8.0 Hz), 5.01-4.97 (dd, 1H, J=10.4, 3.2 Hz), 4.55 (d, 1H, J=8.0 Hz), 4.17-4.07 (m, 2H), 3.96-3.87 (m, 2H), 3.76-3.71 (m, 1H), 3.65-3.61 (m, 4H), 3.35-3.32 (m, 2H), 2.12, 2.04, 2.01, 1.95 (4 s, each 3H). ¹³C NMR (CDCl₃): δ 170.60, 170.46, 170.34, 169.71, 101.54, 71.10, 70.88, 70.64, 70.41, 69.25, 69.02, 67.25, 61.48, 50.98, 20.98, 20.88, 20.80. MS ES⁺ m/z: 484.06 (M+Na).

2-azidoethyl 2,3,4,6-tetra-O-acetyl-β-glucopyranoside (20): ¹H NMR (400 MHz, CDCl₃) δ 5.21-5.17 (t, 1H, J=9.6 Hz), 5.10-5.05 (t, 1H, J=9.6 Hz), 5.02-4.98 (dd, 1H, J=9.2, 8.0 Hz), 4.58 (d, 1H, J=7.2 Hz), 4.25-4.21 (dd, 1H, J=13.2, 4.4 Hz), 4.15-4.12 (dd, 1H, J=12.0, 2.4 Hz), 4.04-3.99 (m, 1H), 3.71-3.64 (m, 2H), 3.51-3.45 (m, 1H), 3.29-3.23 (m, 1H), 2.07, 2.03, 2.01, 1.98 (4 s, each 3H). ¹³C NMR (CDCl₃) δ 170.88, 170.50, 169.62, 100.86, 72.97, 72.10, 71.22, 68.82, 68.46, 61.70, 50.70, 20.97, 20.92, 20.83. MS ES⁺ m/z: 440.22 (M+Na).

2-azidoethyl 2,3,4,6-tetra-O-acetyl-β-lactopyranoside (22): ¹H NMR (400 MHz, CDCl₃): δ 5.29 (d, 1H, J=3.2 Hz), 5.15 (t, 1H, J=9.2 Hz), 5.05 (t, 1H, J=9.2 Hz), 4.94-4.84 (m, 2H), 4.52-4.44 (m, 2H), 4.09-4.03 (m, 4H), 3.96-3.90 (m, 1H), 3.85-3.75 (m, 2H), 3.65-3.59, (m, 2H), 3.45-3.40 (m, 1H), 3.24-3.20 (m, 1H), 2.10 (s, 3H), 2.07 (s, 3H), 2.01 (s, 3H), 1.99 (s, 9H), 1.91 (s, 3H). ¹³C NMR (CDCl₃): δ 170.55, 170.35, 170.26, 169.96, 169.26, 100.28, 100.62, 76.35, 72.98, 72.88, 71.64, 71.14, 70.83, 69.25, 68.91, 66.77, 61.97, 60.97, 50.67, 21.06, 20.99, 20.92, 20.84, 20.72. MS ES⁺ m/z: 728.08 (M+Na).

General deprotection procedure of O-acetyl-pyranoside: The protected pyranoside (200 mg) was dissolved in dry methanol (MeOH) (10 mL). NaOMe solution (1 M in MeOH) was added until pH 9 was reached. Then the reaction mixture was stirred at room temperature until the deprotection reaction was complete (as assayed by TLC iso-PrOH-water, 7:3, +1% NH₃). Then the mixture was neutralized with ion-exchange resin (Amberylst), filtered, and dry in vacuum. The resulting residue was then dissolved in ethanol (EtOH) (10 mL), and then palladium catalyst (10% Pd—C, 50 mg) and few drops of acetic acid were added. The reaction mixture was hydrogenated at 50 psi for 20 hrs. Then it was filtered through a Celite bed, washed with MeOH. The filtrate was concentrated under low pressure to give final product.

The chemical properties of the deprotected O-acetyl-pyranoside are as follows:

2-aminoethyl-β-galactopyranoside acetate (16): ¹H NMR (500 MHz, CD₃OD): δ 4.30 (d, 1H, J=7.5 Hz), 4.06-4.02 (m, 1H), 3.92-3.88 (m, 1H), 3.84 (d, 1H, J=2.5 Hz), 3.78-3.70 (m, 2H), 3.60-3.50 (m, 3H), 3.18-3.15 (m, 1H), 1.93 (s, 3H). ¹³C NMR (CD₃OD): δ 177.63, 103.66, 75.77, 73.55, 71.30, 69.09, 65.76, 61.37, 39.87, 21.99. MS ES⁺ m/z: 246.35 (M+Na).

3-aminopropyl-β-galactopyranoside acetate (17): ¹H NMR (400 MHz, CD₃OD): δ 4.27 (d, 1H, J=7.2 Hz), 4.05-3.99 (m, 1H), 3.83 (d, 1H, J=1.6 Hz), 3.80-3.73 (m, 3H), 3.56-3.46 (m, 3H), 3.14-3.08 (m, 2H), 1.97-1.95 (m, 2H), 1.93 (s, 3H). ¹³C NMR (CD₃OD): δ 103.83, 75.68, 73.71, 71.25, 69.12, 67.61, 61.33, 38.03, 27.18, 21.66. MS ES⁺ m/z: 260.08 (M+Na).

5-aminopentyl-O-galactopyranoside acetate (18): ¹H NMR (500 MHz, CD₃OD): δ 4.22 (d, 1H, J-7.2 Hz), 3.94-3.89 (m, 1H), 3.84 (d, 1H, J=2.4 Hz), 3.72 (d, 2H, J=5.6 Hz), 3.61-3.55 (m, 1H), 3.52-3.46 (m, 3H), 2.94-2.90 (m, 2H), 1.93 (s, 3H), 1.73-1.63 (m, 4H), 1.59-1.42 (m, 2H). ¹³C NMR (CD₃OD): δ 103.79, 75.43, 73.83, 71.42, 69.09, 69.04, 61.27, 39.41, 28.86, 27.20, 27.01, 22.81, 22.70, 21.71. MS ES⁺ m/z: 266.10 (M+H).

5-amino-3-oxapentyl-β-galactopyranoside acetate (19): ¹H NMR (500 MHz, CD₃OD): δ 4.30-4.28 (d, 1H, J=7.5 Hz), 4.05-4.01 (m, 1H), 3.84-3.83 (dd, 1H, J=3.0, 1.0 Hz), 3.80-3.76 (m, 1H), 3.75-3.71 (m, 6H), 3.54-3.47 (m, 3H), 3.13-3.11 (m, 2H), 1.95 (s, 3H). ¹³C NMR (CD₃OD): δ 176.37, 103.82, 75.61, 73.81, 71.36, 69.97, 69.14, 68.90, 66.63, 61.35, 39.37, 21.13. MS ES⁺ m/z: 268.19 (M+H).

2-aminoethyl-β-glucopyranoside acetate (21): ¹H NMR (400 MHz, CD₃OD): δ 4.34 (d, 1H, J=8.0 Hz), 4.08-4.03 (dt, 1H, J=12.0, 4.8 Hz), 3.90-3.84 (m, 2H), 3.69-3.65 (dd, 1H, J=12.0, 5.6 Hz), 3.41-3.22 (m, 4H), 3.17-3.15 (t, 1H, J=6.0 Hz), 1.94 (s, 3H). ¹³C NMR (CD₃OD): δ 176.63, 103.08, 76.91, 76.59, 73.74, 70.27, 65.86, 61.33, 39.73, 21.60. MS ES⁺ m/z: 246.05 (M+Na).

2-aminoethyl-β-lactopyranoside acetate (23): ¹H NMR (300 MHz, CD₃OD): δ 4.39-4.37 (d, 1H, J=8.4 Hz), 4.36-4.35 (d, 1H, J=7.2 Hz), 4.07-4.04 (m, 1H), 3.94-3.68 (m, 6H), 3.62-3.46 (m, 7H), 3.34-3.30 (m, 1H), 3.18-3.16 (t, 1H, J=4.8 Hz), 1.96 (s, 3H). ¹³C NMR (CD₃OD): δ 175.62, 103.92, 102.78, 79.16, 75.93, 75.40, 75.02, 73.59, 73.42, 71.33, 69.09, 65.76, 61.34, 60.45, 39.70, 20.65. MS ES⁺ m/z: 408.01 (M+Na).

General procedure for the synthesis of sugar-GA conjugate through C-17 linkage of geldanamycin: GA (30 mg, 0.54 mmol), sugar-amine derivative (4 eq), and 100 μL Et₃N were dissolved in 0.5 mL dry DMF. The mixture was stirred at room temperature in the dark for 24 hr. Then the mixture was directly subjected to flash chromatography (silica gel, hexane/EtOAc system, then EtOAc/MeOH 9:1) to give pure carbohydrate-GA conjugate.

The chemical properties of the synthesized sugar-GA conjugate are as follows:

17-demethoxy-17-[(2-β-glucopyranosylethyl)amino]geldanamycin (24): ¹H NMR (400 MHz, CD₃OD): δ 7.13 (d, 1H, J=12.4 Hz), 7.05 (s, 1H), 6.62, (t, 1H, J=12.0 Hz), 5.87 (t, 1H, J=8.8 Hz), 5.58 (d, 1H, J=9.6 Hz), 5.20 (s, 1H), 4.53 (d, 1H, J=8.4 Hz), 4.34 (d, 1H, J=8.0 Hz), 4.07-4.01 (m, 1H), 3.92-3.89 (m, 2H), 3.83-3.78 (m, 1H), 3.76-3.70 (m, 2H), 3.60-3.55 (m, 1H), 3.46 (br, 1H), 3.34-3.29 (m, 9H), 3.24-3.19 (t, 1H, J=8.0 Hz), 2.75-2.68 (m, 2H), 2.37-2.32 (dd, 1H, J=14.4, 8.88 Hz), 1.99-1.98 (s, 3H), 1.79 (br, 1H), 1.73 (s, 3H), 1.65 (br, 1H), 1.60-1.56 (m, 1H), 0.99-0.96 (t, 6H, J=7.2 Hz). MS ES⁺ m/z: 774.27 (M+Na).

17-demethoxy-17-[(2-β-galactopyranosylethyl)amino]geldanamycin (25): ¹H NMR (500 MHz, CD₃OD) δ 7.12 (d, 1H, J=11.5 Hz), 7.04 (s, 1H), 6.62, (t, 1H, J=12.0 Hz), 5.86 (t, 1H, J=9.0 Hz), 5.58 (d, 1H, J=10.0 Hz), 5.20 (s, 1H), 4.53 (d, 1H, J=8.5 Hz), 4.30 (d, 1H, J=8.0 Hz), 4.06-4.02 (m, 1H), 3.91-3.87 (m, 1H), 3.84-3.79 (m, 3H), 3.76-3.69 (m, 2H), 3.60-3.55 (m, 3H), 3.50-3.44 (m, 2H), 3.34 (s, 3H), 3.29 (s, 3H), 2.74-2.68 (m, 2H), 2.36-2.31 (dd, 1H, J=14.5, 9.0 Hz), 1.99 (s, 3H), 1.79 (bs, 1H), 1.73 (s, 3H), 1.68-1.65 (br, 1H), 1.60-1.56 (m, 1H), 0.99-0.96 (t, 6H, J=8.5 Hz). MS ES⁺ m/z: 774.29 (M+Na).

17-demethoxy-17-[(2-β-galactopyranosylpropyl)amino]geldanamycin (26): ¹H NMR (500 MHz, CD₃OD) δ 7.13 (d, 1H, J=12.0 Hz), 7.05 (s, 1H), 6.65-6.60 (t, 1H, J=12.0 Hz), 5.89-5.85 (t, 1H, J=11.0 Hz), 5.58 (d, 1H, J=10.0 Hz), 5.22 (s, 1H), 4.54 (d, 1H, J=8.0 Hz), 4.23 (d, 1H, J=7.5 Hz), 4.03-3.98 (m, 1H), 3.84-3.83 (d, 1H, J=3.5 Hz), 3.78-3.67 (m, 5H), 3.60-3.56 (m, 2H), 3.53-3.45 (m, 3H), 3.34 (s, 3H), 3.29 (s, 3H), 2.77-2.68 (m, 2H), 2.39-2.35 (dd, 1H, J=14.0, 9.0 Hz), 1.99 (s, 3H), 1.96-1.93, (m, 2H), 1.79 (br, 1H), 1.73 (s, 3H), 1.70-1.68 (br, 1H), 1.67 (br, 1H), 1.00-0.97 (t, 6H, J=6.5 Hz). MS ES⁺ m/z: 788.26 (M+Na).

17-demethoxy-17-[(2-β-galactopyranosylpentyl)amino]geldanamycin (27): ¹H NMR (500 MHz, CD₃OD) δ 7.13 (d, 1H, J=11.5 Hz), 7.05 (s, 1H), 6.64-6.60 (t, 1H, J=11.5 Hz), 5.89-5.85 (t, 1H, J=11.0 Hz), 5.58 (d, 1H, J=9.5 Hz), 5.21 (s, 1H), 4.54 (d, 1H, J=8.5 Hz), 4.21 (d, 1H, J=7.5 Hz), 3.93-3.90 (m, 1H), 3.82 (d, 1H, 3.5 Hz), 3.74-3.71 (m, 2H), 3.60-3.44 (m, 8H), 3.34 (s, 3H), 3.29 (s, 3H), 2.75-2.73 (m, 2H), 2.35-2.30 (dd, 1H, J=14.0, 9.0 Hz), 1.99 (s, 3H), 1.81 (bs, 1H), 1.73-1.49 (m, 11H), 0.99-0.96 (m, 6H). MS ES⁺ m/z 816.36 (M+Na).

17-demethoxy-17-{[2-(2-β-galactopyranosylethyl)ethyl]amino}geldanamycin (28): ¹H NMR (500 MHz, CD₃OD) δ 7.14-7.12 (d, 1H, J=11.5 Hz), 7.06 (s, 1H), 6.64-6.60, (t, 1H, J=12.0 Hz), 5.87-5.85 (t, 1H, J=9.0 Hz), 5.60-5.58 (d, 1H, J=10.0 Hz), 5.20 (s, 1H), 4.54-4.53 (d, 1H, J=9.0 Hz), 4.29-4.28 (d, 1H, J=7.5 Hz), 4.05-4.01 (m, 1H), 3.83-3.82 (m, 1H), 3.78-3.70 (m, 9H), 3.61-3.28 (t, 1H, J=5.5 Hz), 3.56-3.45 (m, 4H), 3.34 (s, 3H), 3.29 (s, 3H), 2.75-2.69 (m, 2H), 2.36-2.32 (dd, 1H, J=14.5, 9.5 Hz), 1.99 (s, 3H), 1.81 (bs, 1H), 1.73 (s, 3H), 1.68-1.65 (br, 1H), 1.60-1.56 (m, 1H), 1.00-0.98 (d, 3H, J=6.5 Hz), 0.98-0.96 (t, 3H, J=7.0 Hz). MS ES⁺ m/z: 818.49 (M+Na).

17-demethoxy-17-[(2-β-lactopyranosylethyl)amino]geldanamycin (29): ¹H NMR (500 MHz, CD₃OD): δ 7.12 (d, 1H, J=11.5 Hz), 7.05 (s, 1H), 6.64-6.60 (t, 1H, J=12.0 Hz), 5.89-5.85 (t, 1H, J=9.0 Hz), 5.58 (d, 1H, J=110.0 Hz), 5.21 (s, 1H), 4.53 (d, 1H, J=8.0 Hz), 4.39-4.37 (m, 3H), 4.04-3.99 (m, 1H), 3.94-3.87 (m, 3H), 3.83-3.73 (m, 3H), 3.71-3.66 (m, 2H), 3.61-3.52 (m, 4H), 3.50-3.44 (m, 4H), 3.34 (s, 3H), 3.29 (s, 3H), 2.74-2.68 (m, 2H), 2.36-2.32 (dd, 1H, J=14.0, 8.5 Hz), 1.99 (s, 3H), 1.80 (br, 1H), 1.73 (s, 3H), 1.68-1.65 (br, 1H), 1.60-1.56 (m, 1H), 0.99-0.96 (t, 6H, J=8.5 Hz). MS ES⁺ m/z: 936.28 (M+Na).

1-deoxy-2,3,4,6-tetrakis-O-phenylmethyl-1-phenylsulfinyl-mannopyranose (30): mCPBA (75%, 0.36 g, 1 eq) in CH₂Cl₂ (100 mL) was dropwise added to a solution of sulfide (1.0 g, 1.58 mmol) in 25 mL CH₂Cl₂ at −78° C. during 30 min. The mixture was warmed to 0° C. over 1 hr, quenched with saturated NaHCO₃, and extracted with CH₂Cl₂. The combined organic phase was dried over Na₂SO₄, filtered, concentrated, and purified by flash chromatography to give the product as two diasteremers. Isomer A: ¹H NMR (500 MHz, CDCl₃) δ 7.66-7.11 (m, 25H), 5.04-4.98 (q, 2H, J=10.0 Hz), 4.93-4.91 (d, 1H, J=12.0 Hz), 4.77-4.71 (q, 2H), 4.64-4.61 (d, 1H, J=12.0 Hz), 4.50-4.47 (t, 1H, J=9.5 Hz), 4.15 (s, 2H), 3.95-3.93 (d, 1H, J=110.0 Hz), 3.89 (d, 1H, J=3.0 Hz), 3.70-3.67 (dd, 1H, J=9.5, 2.5 Hz), 3.52-3.48 (m, 1H), 3.43-3.38 (m, 2H). ¹³C NMR (CDCl₃) δ 140.32, 138.61, 138.18, 138.07, 131.00, 128.95, 128.75, 128.72, 128.55, 128.53, 128.47, 128.23, 128.07, 128.02, 127.95, 127.86, 127.82, 125.52, 94.30, 84.50, 79.33, 76.22, 74.59, 74.06, 73.63, 73.29, 72.84, 69.21. MS ES⁺ m/z: 649.19 (M+H). Isomer B: ¹H NMR (500 MHz, CDCl₃) δ 7.62-7.12 (m, 25H), 4.92-4.84 (m, 3H), 4.74-4.64 (q, 2H), 4.53-4.50 (m, 2H), 4.47-4.40 (q, 2H), 4.10-4.06 (t, 1H, J=9.5 Hz), 3.97 (s, 1H), 3.74-3.66 (m, 3H), 3.57-3.54 (dd, 1H, J=9.0, 7.0 Hz). ¹³C NMR (CDCl₃): δ 140.47, 138.83, 138.20, 138.03, 138.00, 131.27, 128.98, 128.80, 128.76, 128.72, 128.54, 128.40, 128.20, 128.15, 128.10, 128.05, 127.91, 127.56, 127.45, 126.23, 95.65, 84.11, 77.73, 74.71, 74.31, 73.87, 73.80, 72.90, 72.56, 68.34. MS ES⁺ m/z: 649.20 (M+H).

17-demethoxy-17-amino-geldanamycin (34): ¹H NMR (500 MHz, CDCl₃) δ 9.08 (s, 1H), 7.23 (s, 1H), 6.92 (d, 1H, J=11.5 Hz), 6.58-6.53 (t, 1H, J=11.5 Hz), 5.90-5.88 (d, 1H, J=9.5 Hz), 5.86-5.82 (t, 1H, J=110.5 Hz), 5.523 (br, 1H), 5.14 (s, 1H), 4.28 (d, 2H, J=10.0 Hz), 3.65 (t, 1H, J=7.5 Hz), 3.44-3.41 (m, 1H), 3.35 (s, 3H), 3.26 (s, 3H), 2.77 (t, 1H, J=7.0 Hz), 2.71 (d, 1H, J=13.5), 2.02 (s, 3H), 2.00-1.95 (m, 1H), 1.88-1.76 (m, 6H), 1.01-0.97 (m, 6H). MS ES⁺ m/z: 568.32 (M+Na).

### EXAMPLE 3

### Tumor Targeting, Pharmacokinetics, and Immunogenicity of Humanized TAG-72 Antibody (CC49ΔC_{H}2) in Colorectal Cancer Patients

TABLE 3
Mean precordial counts on the day of surgery

| | Interval from | Mean Precordial |
|---|---|---|
| | Injection to | Count: Day of |
| | Surgery | Surgery |
| Patients | (days) | (count/2 sec) |
| 1 | 3 | 1879 |
| 2 | 3 | 950 |
| 3 | 3 | 1322 |
| 4 | 5 | 438 |
| 5 | 5 | 980 |
| 6 | 5 | 184 |
| 7 | 7 | 198 |
| 8 | 7 | 150 |
| 9 | 7 | 96 |
| 10 | 9 | 143 |
| 11 | 9 | 233 |
| 12 | 9 | 80 |
| 13 | 11 | 125 |
| 14 | 11 | 688 |
| 15 | 13 | 182 |
| 16 | 15 | 36 |
| 17 | 19 | 8 |
| 18 | 20 | 28 |
| 19 | 20 | 14 |
| 20 | 21 | 18 |
| 21 | 20 | 25 |

Radiolabeled murine B72.3 Mab has been shown to be effective in localizing gastrointestinal tract and prostate tumors. However, it also induce host anti-mouse antibodies (HAMA) in the human population. To limit the HAMA response, the anti-TAG-72 monoclonal antibody CC49 was humanized by grafting of the murine antibody complementary determining regions (CDRs) onto the frameworks of human antibodies. While humanization of mAbs may decrease the potential for antigenicity in human patients, it does not reduce the overall size of the constituent molecules. In order to reduce its molecular weight, modification of the HuCC49 molecule has been performed by deleting the C_{H}2 domain. HuCC49ΔC_{H}2 (C_{H}2 domain deleted mAb) has been shown to have a faster elimination rate and more rapid tumor targeting than HuCC49 without the deletion. It is not known whether the loss of the C_{H}2 glycosylation site affects the tumor targeting and pharmacokinetics of the antibody.

To assess the safety and adverse reactions associated with use of HuCC49ΔC_{H}2, tumor localization, antibody elimination rate, and HAMA production were measured. A pilot study was carried out with radiolabeled HuCC49ΔC_{H}2 in 21 patients undergoing RIGS with recurrent or metastatic colorectal carcinoma. The patient population included 12 males and 9 females with an average age of 56 years old (range, 41-74 years). The colon was the primary carcinoma site in 12 patients and the rectum was the primary site in 9 patients. Three patients had primary colorectal disease at the time of surgery, and 18 had recurrent disease. The median interval from diagnosis of the primary disease and entry into this study was 3 years (range 0-12 years). Seven patients had radiotherapy and 13 patients had chemotherapy prior to surgery.

After thyroid blockade, 1 mg of HuCC49ΔC_{H}2 radiolabeled with 2 mCi of ¹²⁵I was administered to the patients. Daily precordial counts were performed using a gamma-detecting probe. The radioactivity in tumors and normal tissues was measured. Each patient underwent traditional exploration 3 to 21 days after injection, and the blood pool background (BPB) was determined during surgery. The precordial counts and BPB were used to determine the elimination rate of HuCC49ΔC_{H}2. To determine the location in the abdomen that provided the best correlation with precordial counts, the BPB was measured intraoperatively at four locations: suprapancreatic [SP] area, aorta/vena cava [AV], infrapancreatic [IP] area, and aortic bifurcation [AB]. In this manner, we could achieve a desired BPB by examining the decay curve based on precordial counting. Correlation coefficients between the precordial counts and BPB counts from the four areas were 0.96, 0.92, 0.96, and 0.97, for SP, AV, IP, and AB, respectively. Based on these counts, we used precordial counts to predict BPB levels.

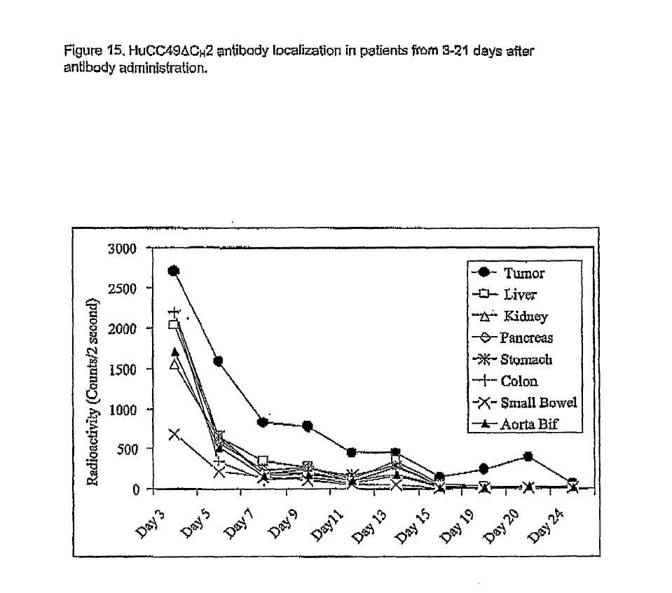

Localization of tumor. The radiolabeled mAb localized a clinically evident recurrent tumor in all but one patient. This patient was initially diagnosed with rectal carcinoma but was found to have a primary neuroendocrine tumor rather than rectal recurrence. While all patients demonstrated detection of grossly evident tumor, the initial cohort of patients with an interval of 3 days from injection to surgery showed the background level of radiation to be too high to provide an optimal tumor-to-background ratio necessary to distinguish small, non-clinically-evident occult lesions. The BPB were too high to allow accurate probe results when there was a short interval between mAb injection and surgery. This required increasing the time interval from injection to surgery in the subsequent cohort of patients until an appropriate time interval could be established. At approximately 20 days from injection to surgery, the BPB was reduced to a level that allowed the detection of occult disease in five of five patients. In all five patients, precordial counts were less than 30 counts per 2 seconds on the day of surgery. Patients in prior cohorts had precordial counts greater than 30 counts and these patients were found to have BPB that were too high to allow accurate gamma probe occult tumor detection (Table 3). However, the antibody has demonstrated the consistent higher tumor localization than any other normal tissues including liver, kidney, pancreas, stomach, colon, small intestine, and aorta bifurcation in patients after antibody administration from 3-21 days (FIG. 15). Interestingly, after 15 days post antibody administration, the radioactivity in normal tissues became undetectable, while tumor tissue still show distinguishable radioactive counts to allow precise surgery.

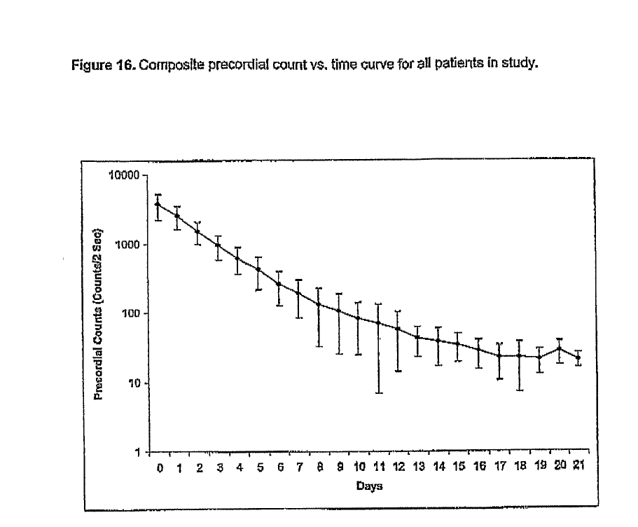

Elimination rate of HuCC49ΔC_{H}2. The monoclonal antibody elimination rates were calculated with WinNonlin for all patients as seen in FIG. 15. The C₀ was 3589±1529 count/2 sec. The mean elimination rate constant was 0.451±0.110 days⁻¹, corresponding to a mean half-life of 1.63±0.41 days with observed half-lives ranging from 1.00 to 2.59 days, which is similar to the murine mAb CC49. A composite precordial count vs. time curve for all patients is presented in FIG. 16.

HAMA levels. In all subjects, human anti-mouse antibody [HAMA] levels were measured pre- and post-injection of HuCC49ΔC_{H}2. The HAMA results were negative (<100 ng/ml) and are summarized in Table 4. The humanized HuCC49ΔC_{H}2 generated no HAMA response in any of the 21 patients.

In summary, no significant difference was observed in elimination rate and tumor localization between murine CC49 and humanized HuCC49ΔC_{H}2. Since extensive studies has been performed for murine CC49 mAb in tumor targeting and RIGS in patients, the intact murine mAb CC49 can be used for the conjugation of CC49 with alpha-galactosidase for targeting alpha-galactosidase to tumors.

TABLE 4
Human anti-mouse antibody (HAMA) Levels pre- and post-injection
with the HuCC49ΔC_{H}2 MAb in 21 patients.

| HAMA Baseline levels | HAMA at 4-6 weeks | HAMA at 12 weeks |
|---|---|---|
| (ng/mL, n = 21) | (ng/mL, n = 21) | (ng/mL, n = 21) |
| 15.3 ± 5.8 | 16.3 ± 10.8 | 16.1 ± 10.3 |

Chemical conjugation of CC49 with alpha-galactosidase: The glycosylation site of antibody CC49 was chosen for conjugation with alpha-galactosidase. The glycosylation site in CC49 allows for site-specific chemical conjugation of the antibody with the enzyme to minimize the loss of antibody binding affinity to TAG-72. Random conjugation of antibody CC49 with alpha-galactosidase could potentially result in the linkage of the enzyme at Fab′ portion of the antibody, thus reducing net binding affinity to the antigen of a conjugated antibody population. Conjugation can be carried out at a specific location, such as the glycosylation site, in order to minimize steric interference effects of the conjugated enzyme. The glycosylation site of the antibody is located in the C_{H}2 portion of the heavy chain. The Examples above demonstrate that deletion of this glycosylation sites does not affect the antibody tumor targeting specificity and elimination rate. Based on these experiments, the CC49-alpha-galactosidase antibody-enzyme complex is expected to localize specifically at the surface of the TAG-72 positive tumor cells. This targeted alpha-galactosidase, not present in blood circulation, will site-specifically activate carbohydrate conjugated geldanamycin prodrugs in tumors to achieve better tumor targeting efficacy and lower tissue toxicity.

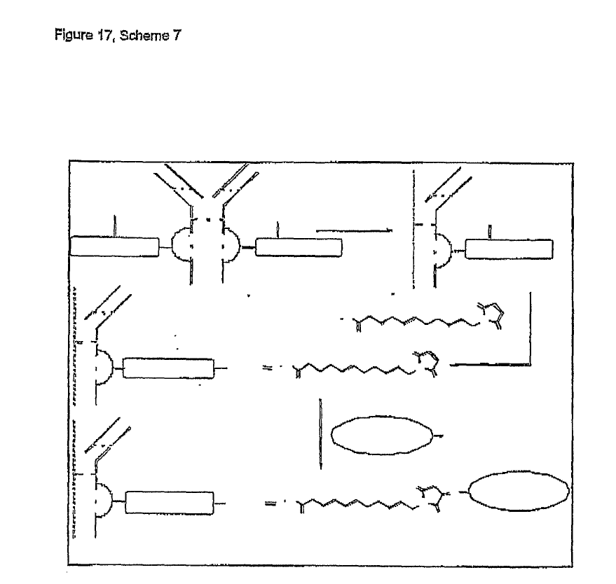

Antibody CC49 can be incubated with neuraminidases to cleave Gal-α-(2→6)-NANA and Gal-α-(2→3)-NANA bonds, producing fully desialyated carbohydrate moieties, making accessible the Gal residues by galactose oxidase (GAO). GAO can convert primary alcohols of galactopyranosyl residues to the corresponding aldehyde groups, which will be further transformed into Schiff bases with KMUH (N-[κ-Maleimidoundecanoic acid]hydrazide). The presence of galactose acceptor sites of the antibody will be determined based on a GAO-toluidine-horseradish peroxides assay. Alpha-galactosidase is a thiol-containing protein chosen to react with the maleimide group of the above Schiff base to prepare conjugated antibody-protein complexes. The enzyme-antibody conjugation procedure is depicted in FIG. 17, Scheme 7. Thus, alpha-galactosidase will be attached to antibody CC49 carbohydrate region, avoiding blockage at the antibody active site. Alternatively, the invention can be practiced by adding an active thiol group through a reaction of alpha-galactosidase and 2-Iminothiolane to increase the reactivity of alpha-galactosidase for forming antibody-enzyme conjugates.

### EXAMPLE 4

### Enzyme-Specific Activation of Geldanamycin-Carbohydrates Prodrugs with CC49-Alpha-Galactosidase in LS 174T Colorectal Adenocarcinoma Cells In Vitro

Anticancer activity of geldanamycin-carbohydrate prodrugs in combination with CC49-alpha-galactosidase can be tested with the MTS assay (MTS: tetrazolium [3-(4, 5-dimethylhiazol-2-yl)]-5-(3-carboxymethoxyphenyl)-2-(4-sulfophenyl)-2H-tetrazolium) in colorectal adenocarcinoma cells LS 174T. This can demonstrate whether CC49-alpha-galactosidase (1) still maintains the binding activity to TAG72 in cancer cells; and (2) is still active to cleave the prodrug with alpha-galactoside linkage in cell culture. The LS 174T cell line is useful for performing this assay due to its high expression levels of TAG-72. A total of 2,000-5,000 LS 174T cells are seeded in 96-well plates and incubated for 24 hours. The CC49-alpha-galactosidase (equivalent to two units of enzyme) is added to the cell culture to allow antibody binding to TAG-72 displayed on cancer cells. After one hour, the cells are washed three times with culture medium to remove unbound antibody-enzyme complexes. Synthesized prodrugs are then added using a series of dilutions and incubated for 4 days. As a positive control, alpha-galactosidase (2 units) is incubated with the synthesized prodrug compounds. As a negative control, synthesized prodrug alone are incubated with cells in the absence of enzyme or antibody-enzyme complex. After 4 days, MTS (2 mg/ml) and phenazine methosulfate (PMS, 25 μM) are added directly to the cell culture and incubated for 1-2 hours at 37° C. The absorbance of formazan (the metabolite of MTS in viable cells) is measured at 490 nm to quantify the number of surviving cells. The IC₅₀ values of the tested compounds are calculated using dose response curves. In addition, similar experiments can be carried out utilizing other colon cancer cells with low expression of TAG-72 (e.g., SW-620 or HT-29). It is expected that just as geldanamycin-beta-galactose is cleaved by beta-galactosidase, geldanamycin-alpha-galactose prodrug will be cleaved and activated by alpha-galactosidase. Therefore, if LS 174T cells are pretreated with CC49-alpha-galactosidase, or co-incubated with alpha-galactosidase, and synthesized conjugated prodrug compounds are present, then these prodrugs will be activated to exhibit anticancer activity. Prodrug alone is not expected to produce any anticancer activity. On the other hand, the synthesized prodrugs are not expected to show anticancer activity in colon cancer cells (SW-620 or HT 29) even with the pre-treatment of CC49-alpha-galactosidase, because when the TAG-72 antigen is absent, the unbound antibody-enzyme complexes will not accumulate on the cells. Since the chemical conjugation of CC49 antibody and alpha-galactosidase is carried in mild conditions, no loss of binding activity of the antibody or enzymatic activity of alpha-galactosidase is expected. Since alpha-galactosidase is a much larger molecule than CC49 antibody, blockage of the Fab portion of the antibody by the enzyme, can be avoided by site-specific conjugation with the glycosylation site in the C2H chain. The loss of the glycosylation site of the antibody did not affect antibody binding affinity and pharmacokinetic profiles.

### EXAMPLE 5

### Targeted Delivery of CC49-Alpha-Galactosidase to Tumor Sites and Site-Specific Activation of Geldanamycin-Carbohydrate Prodrugs in Xenograft Mouse Models

Results have shown that murine and humanized anti-TAG antibody is able to localize and target about 80% of colon cancer tissues in human. Validation of CC49 antibody-alpha-galactosidase localization to colon cancer cells can be tested in xenograft mouse models.

Although in vitro models were used to examine the molecular aspects of drug activity and transport, the predicted effects on animal physiology, including those in humans, (e.g., absorption, distribution, metabolism, and excretion) and efficacy in vivo, can be further validated by utilizing an in vivo model system. The nude mouse xenograft model is an established animal model for studying human cancer in vivo. This strain of mice can form the tumors with human cancer cells due to its immunodeficiency and is also relatively inexpensive.

The colorectal adenocarcinoma cells LS 174T (expressing high levels of TAG-72) is used to establish the xenograft models and can be compared with colorectal carcinoma cells SW-620 (expressing low levels of TAG-72). Cancer cells (10⁶ cells) in matrigel/medium (100 μl) are injected subcutaneously or intaperitoneally into nude mice. Tumors are allowed to grow for two weeks, with tumor volumes measured twice per week with calculated tumor volume [(W²×L)/2]. When tumors reach 100 mm³, animals are randomized treatment groups. The CC49-alpha-galactosidase is administered to test mice through tail veil injection. At different time points (2 hr, 1 day, 3 days, 7 days, 13 days, 21 days), the mice are sacrificed to allow collection of tumor tissues. The distribution of the CC49-alpha-galactosidase can be monitored by either immunohistochemistry or western blotting. For immunohistochemistry, the tumor tissue is sectioned and stained with FITC labeled goat-anti-human IgG to allow CC49 detection, or incubated with primary antibody for alpha-galactosidase and a FITC-labeled secondary antibody to allow galactosidase detection. For western blotting, the tumor tissue is homogenized in lysis buffer with trypsin inhibitor and phenylmethylsulfonyl fluoride (PMSF). Following blotting, the blot is probed with anti-human IgG or anti-alpha-galactosidase antibody. Chemilluminesence or other means known to artisans can be used for detection.

Following validation of TAG-72 binding affinity and enzymatic activity of the CC49-alpha-galactosidase complex, in situ targeting of the antibody-enzyme complex should be much more efficient with LS 174T xenografts, and similar to the unmodified CC-49 antibody alone, but the SW-620 cancer cell xenografts, with low expression levels of TAG-72, are not expected to accumulate CC49-alpha-galactosidase complexes. The distribution of antibody-enzyme complexes in normal tissues will be much lower than in tumor tissues, and will decrease rapidly over time. Utilizing a sampling range of from 2 hrs to 21 days to analyze the tumor targeting allows assessment of antibody distribution in normal tissues before day 13 (although antibody complexes preferentially accumulate in the tumor tissues). At 21 days, the tumor tissue will retain high antibody distribution while other normal tissues are expected to have undetectable antibody distribution. Following an initial in situ targeting assay, it is possible to choose one or two time points for the subsequent assays to optimize dosing and timing necessary to achieve specific activation of conjugated prodrugs in cancer tissue.

The localization of CC49-alpha-galactosidase complex at tumor sites along with cleavage and activation geldanamycin-galactose prodrugs in vivo will enable improved efficacy of anti-cytostatic treatment and lower tissue toxicity. The improvement of activity can be validated by utilizing mouse xenograft models. Xenograft models, as described above, with LS 174T or SW-620 cells can be used. When the implanted tumor size reaches 100 mm³, animals are randomized into treatment groups. The CC-49-alpha-galactosidase complex is administered through tail vein injection. After a chosen time point (based on the results of the in situ targeting assay identifying time points with the high accumulation of antibody-enzyme complex in tumors and minimum residual accumulation in normal tissues), mice are treated with geldanamycin-galactose prodrugs or geldanamycin alone through intravascular administration. Tumor growth can be assessed by measuring tumor volume once every 3 days. The mouse survival rate is calculated based on the surviving number of animals, including animals euthanized if early removal criteria are met. Thus, tumor localized CC49-alpha-galactosidase can cleave and activate geldanamycin-galactose prodrug at the tumor site. Therefore, the LS 174T xenograft model, displaying the TAG-72 antigen, the geldanamycin-galactose treatments are expected to show improved efficacy for inhibiting tumor growth and increased survival rate over unconjugated geldanamycin in combination with CC49-alpha-galactosidase. More importantly, the carbohydrate conjugated prodrug is expected to have lower systemic toxicity and a better therapeutic index over geldanamycin. Conversely, in the SW-620 xenograft models, the CC49-alpha-galactosidase will not be specifically localized at tumor sites due to the absence of the TAG-72 antigen. Therefore, carbohydrate conjugated geldanamycin prodrugs are expected to show reduced efficacy compared to geldanamycin alone, due to the absence of specific activation by alpha-galactosidase.

Since certain changes may be made in the above compositions and methods without departing from the scope of the invention herein involved, it is intended that all matter contained in the above descriptions and examples or shown in the accompanying drawings shall be interpreted as illustrative and not in a limiting sense. All terms not specifically defined herein are considered to be defined according to Dorland's Illustrated Medical Dictionary, 27^{th} edition, or if not defined in Dorland's dictionary then in Webster's New Twentieth Century Dictionary Unabridged, Second Edition. The disclosures of all of the citations provided are being expressly incorporated herein by reference.

We claim:

1. A method for synthesizing and activating prodrugs, comprising:
(a) synthesizing a prodrug by conjugating a carbohydrate group to an active form of a drug;
(b) administering the prodrug to a patient in need of therapy; and
(c) exposing the prodrug to an agent that can cleave the carbohydrate group.

2. The method of claim 1, wherein said carbohydrate is glucose.

3. The method of claim 1, wherein said carbohydrate is galactose.

4. The method of claim 1, wherein said carbohydrate is lactose.

5. The method of claim 1, wherein said carbohydrate is chosen from mannose, fucose, n-acetylglucosamine, xylose, sialic acid, and glucuronic acid.

6. The method of claim 1, wherein said drug is chosen from paclitaxel, 9-aminocampthotecin, 5-fluorouracil, aniline-mustard, epirubicine, daunorubicin, doxorubicin, beta-naphthol, geldanamycin, cisplatin, nitrogen mustard, indolocarbazole, anthracyclines, SAHA, or rapamycin.

7. The method of claim 1, wherein said prodrug is activated by one or more of acetylase, glucosidase, galactosidase, manosidase, glucoamylase, glucosaminidase, galactosaminidase, sialidase, xylosidase, or glucuronidase.

8. The method of claim 6 wherein the drug is paclitaxel.

9. The method of claim 6 wherein the drug is 5-fluorouracil.

10. The method of claim 6 wherein the drug is cisplatin.

11. The method of claim 6 wherein the drug is geldanamycin.

12. The method of claim 6 wherein the drug is SAHA.

13. The method of claim 7 wherein the enzyme is beta-glucosidase.

14. The method of claim 7 wherein the enzyme is alpha-galactosidase.

15. The method of claim 7 wherein the enzyme is beta-galactosidase.

16. The method of claim 7 wherein the enzyme is alpha-glucosidase.

17. A method of treating cancer, comprising:
(a) administering a carbohydrate-conjugated prodrug to a patient in need of therapy;
(b) providing a means for releasing the carbohydrate from the prodrug; and
(c) allowing the prodrug to be activated.

18. The method of claim 17, wherein said means of releasing the carbohydrate group is a native enzyme enriched in cancer cells.

19. The method of claim 17, wherein said means of releasing the carbohydrate group is an enzyme linked to an antibody directed against a cancer cell antigen.

20. The method of claim 19, wherein the enzyme linked to the antibody is beta-galactosidase.

21. The method of claim 17, wherein said carbohydrate group is glucose and said enzyme is native glucosidase.

22. The method of claim 17, wherein said carbohydrate group is galactose and said enzyme is native glucosidase.

23. A composition of matter comprising 17-demethoxy-17-[(2-β-glucopyranosylethyl)amino]geldanamycin.

24. A composition of matter comprising 17-demethoxy-17-[(2-β-galactopyranosylethyl)amino]geldanamycin.

25. A composition of matter which comprising 17-demethoxy-17-[(2-β-galactopyranosylpropyl)amino]geldanamycin.

26. A composition of matter which comprises one or more of 17-demethoxy-17-[(2-β-galactopyranosylpentyl)amino]geldanamycin; 17-demethoxy-17-{[2-(2-β-galactopyranosylethyl)ethyl]amino}geldanamycin; 17-demethoxy-17-[(2-β-lactopyranosylethyl)amino]geldanamycin.
